SEQUENCE LISTING (13 sequences)

SEQ ID NO: 1 - protein, 40 aa, synthetic construct
  VARIANT 3: note = X = E or D or K or Q
  VARIANT 5: note = X = S or T
  VARIANT 6: note = X = F or Y
  VARIANT 7: note = X = S or T
  VARIANT 8: note = X = S or A or D or E or G or K or N or Q or T
  VARIANT 9: note = X = D or P or E
  VARIANT 10: note = X = L or F or I or M or V
  VARIANT 11: note = X = S or A or D or E or G or K or N or Q or T
  VARIANT 12: note = X = K or E or Q or R
  VARIANT 13: note = X = Q or E or K or R
  VARIANT 14: note = X = M or R or L or V
  VARIANT 15: note = X = E or D or K or Q
  VARIANT 16: note = X = E or D or K or Q
  VARIANT 17: note = X = E or D or K or Q
  VARIANT 18: note = X = A or G or S or T or V
  VARIANT 19: note = X = A or I or L or M or T or V
  VARIANT 20: note = X = R or K or Q
  VARIANT 21: note = X = L or F or I or M or V
  VARIANT 22: note = X = F or Y
  VARIANT 23: note = X = I or G or L or F or M or V
  VARIANT 24: note = X = E or D or K or Q
  VARIANT 26: note = X = L or F or I or M or V
  VARIANT 27: note = X = K or E or Q or R
  VARIANT 32: note = X = S or A or D or E or G or K or N or Q or T
  VARIANT 33: note = X = S or A or D or E or G or K or N or Q or T
  VARIANT 35: note = X = A or G or S or T or V
  VARIANT 39: note = X = S or A or D or E or G or K or N or Q or T
HGXGXXXXXXXXXXXXXXXXXXXXWXXNGGPXXGXPPPXC

SEQ ID NO: 2 - protein, 40 aa, synthetic construct
  VARIANT 9: note = X = D or P or K
  VARIANT 19: note = X = A or T or V
  VARIANT 23: note = X = I or G
  VARIANT 35: note = X = A or G or H or T or R or N
HGEGTFTSXLSKQMEEEAXRLFXEWLKNGGPSSGXPPPSC

SEQ ID NO: 3 - protein, 40 aa, synthetic construct
HGEGTFTSDLSKQMEEEAVRLFIEWLKNGGPSSGAPPPSC

SEQ ID NO: 4 - protein, 40 aa, synthetic construct
HGEGTFTSDLSKQMEEEATRLFIEWLKNGGPSSGAPPPSC

SEQ ID NO: 5 - protein, 40 aa, synthetic construct
HGEGTFTSDLSKQMEEEAARLFIEWLKNGGPSSGAPPPSC

SEQ ID NO: 6 - protein, 40 aa, synthetic construct
HGEGTFTSDLSKQMEEEAVRLFGEWLKNGGPSSGAPPPSC

SEQ ID NO: 7 - protein, 40 aa, synthetic construct
HGEGTFTSPLSKQMEEEAVRLFGEWLKNGGPSSGAPPPSC

SEQ ID NO: 8 - protein, 40 aa, synthetic construct; identical to SEQ ID NO: 3
  SITE 40: note = 3-arm branched Polyethylene glycol (NPEG50K) conjugated to C40

SEQ ID NO: 9 - protein, 40 aa, synthetic construct; identical to SEQ ID NO: 3
  SITE 40: note = 3-arm branched Polyethylene glycol (JPEG50K) conjugated to C40

SEQ ID NO: 10 - protein, 40 aa, synthetic construct; identical to SEQ ID NO: 4
  SITE 40: note = 3-arm branched Polyethylene glycol (JPEG50K) conjugated to C40

SEQ ID NO: 11 - protein, 40 aa, synthetic construct; identical to SEQ ID NO: 5
  SITE 40: note = 3-arm branched Polyethylene glycol (JPEG50K) conjugated to C40

SEQ ID NO: 12 - protein, 40 aa, synthetic construct; identical to SEQ ID NO: 6
  SITE 40: note = 3-arm branched Polyethylene glycol (JPEG50K) conjugated to C40

SEQ ID NO: 13 - protein, 40 aa, synthetic construct; identical to SEQ ID NO: 7
  SITE 40: note = 3-arm branched Polyethylene glycol (JPEG50K) conjugated to C40

(19) United States
(12) Patent Application Publication
(10) Pub. No.: US 2026/0295054 A1
(43) Pub. Date: Oct. 1, 2026

(54) GLUCAGON-LIKE PEPTIDE-1 RECEPTOR AGONIST VARIANTS AND METHODS THEREOF

(71) Applicant: Neuraly, Inc., Gaithersburg, MD (US)

(72) Inventors: Yen Huei LIN, Gaithersburg, MD (US); Jaehee SHIN, Gaithersburg, MD (US); Subramanya KARANTH, Gaithersburg, MD (US); Adam BELL, Gaithersburg, MD (US); Viktor ROSCHKE, Gaithersburg, MD (US)

(21) Appl. No.: 19/479,324

(22) PCT Filed: Apr. 30, 2024
(86) PCT No.: PCT/US2024/027019

§ 371 Date: Oct. 28, 2025

Related U.S. Application Data
(60) Provisional application No. 63/499,395, filed on May 1, 2023.

(51) Int. Cl. A61K 47/60; A61K 9/00; A61K 38/00; A61P 3/04; A61P 3/10; C07K 14/605

(52) U.S. Cl. CPC A61K 47/60; A61K 9/0019; A61P 3/04; A61P 3/10; C07K 14/605; A61K 38/00

(57) ABSTRACT

Glucagon-like peptide-1 (GLP-1) receptor agonists and analogs thereof having one or more amino acids substituted to reduce immunogenicity and antigenicity are described. These GLP-1R agonists and analogs have comparable or better physicochemical properties and pharmacokinetics in a mammalian host relative to GLP-1 RAs currently available for treatment. Compositions and formulations of these agonist peptides are particularly suited for treating, alleviating, and/or preventing one or more metabolic diseases such as obesity, diabetes mellitus, and non-alcoholic fatty liver disease, as well as one or more neurodegenerative disease such as Alzheimer's disease (AD) and Parkinson's disease (PD).

### CROSS-REFERENCE TO RELATED APPLICATION

The present application claims priority to U.S. Provisional Patent Application No. 63/499,395, filed May 1, 2023, the contents of which are hereby incorporated by reference in their entirety.

### FIELD OF THE INVENTION

The invention is generally in the field of custom-designed molecules for treatment of metabolic disorders, and specifically in the area of PEGylated exenatide analogs engineered for low immunogenicity.

### REFERENCE TO SEQUENCE LISTING

The Sequence Listing XML submitted as a file named “NEUR_112_PCT_ST26.xml,” created on Apr. 19, 2024, and having a size of 26,843 bytes is hereby incorporated by reference pursuant to 37 C.F.R. § 1.834 (c) (1).

### BACKGROUND OF THE INVENTION

Glucagon-like peptide-1 receptor agonists (GLP1 RAs) are important therapeutic agents for controlling blood glucose and A1c levels in type 2 diabetes patients after metformin 1st line treatment (ADA Pharmacologic Approaches to Glycemic Treatment; Standards of Medical Care in Diabetes, American Diabetes Association 2020). Recent clinical studies also demonstrate GLP1 RAs such as semaglutide, liraglutide, and tirzepatide are effective in the treatment of obesity, reducing body weight in obese patients with additional benefits in weight-related comorbid condition (e.g., hypertension, type 2 diabetes mellitus, or dyslipidemia).

Most marketed GLP-1 RAs are administered by subcutaneous injection daily (exenatide/BYETTA, liraglutide/VICTOZA and SAXENDA; lixisenatide/ADLYXIN) or once-weekly (albiglutide/TANZIUM; dulaglutide/TRULICITY; exenatide extended release/BYDUREON; semaglutide/OZEMPIC and WEGOVY). Newer formulations are taken by mouth once daily (semaglutide/RYBELSUS). Once-weekly treatments are currently most often prescribed followed by daily treatments. The total cost of treatment accumulates given that treatment for diabetes and obesity are chronic and often long-term. Reducing administration frequency to once or twice a month has several advantages to the patient and payers. Because a monthly treatment is given as 12 doses per year and a weekly treatment is given as 52 doses per year, there can be a significant cost savings in less frequent administration. In addition, patient compliance with their treatment plan and persistence with treatment past an initial treatment phase has been shown to correlate with dosing frequency. As treatments for diabetes, osteoporosis, bipolar disorder, and HIV have been reformulated to extend the interval between doses (from daily to weekly to monthly), compliance with treatment has increased and discontinuation from treatment has decreased. In diabetes, increased adherence and persistence have been shown to improve efficacy.

Compliance and persistence rates with weekly GLP-1RAs are better than daily formulations, but in large real-world studies compliance and persistence were still only at 40-50%. Poor compliance was associated with reduced effectiveness in glucose control and reduced weight loss.

GLP-1R agonists are immunogenic with 30-50% of treated patients developing antibodies. This immune response to GLP-1RA is associated with reduced effectiveness in some patients and can also be associated with injection site reactions or, in some cases, a systemic allergic response. Studies with BYETTA® showed that 38% of treated patients developed anti-exenatide antibodies, 6% with high antibody titers (Bendicho-Lavilla C. et al., Acta Pharm Sin B. 2022 February; 12(2): 621-636). Glycemic control failed in 3% patients. Immunogenicity rates were higher for once weekly exenatide (BYDUREON®) with 43%-49% of patients developing antibodies and 6% with an attenuated glycemic response.

It has been demonstrated that site specific pegylation can be achieved by adding a cysteine at the 40^{th} amino acid residue position of exenatide-based peptides. These may be used as therapeutic agents in treatment of diabetes, obesity, and neurodegenerative diseases such as Parkinson's and Alzheimer's disease (US201910406230 and US20180369340A1). These long-acting exenatide analogues, including site-specific pegylation with a 3-arm branched PEG attached to the C-terminus of the peptide, i.e., Exenatide-Cys-NPEG50K (NLY01), maintain bioactivity while achieving an extended serum half-life of 12 days in humans. Exenatide-Cys-NPEG50K (NLY01) has been used in human clinical trials in healthy volunteers (A Phase 1 Study to Assess the Safety of NLY01 in Healthy Subjects (NLY01-H1); ClinicalTrials.gov Identifier: NCT03672604), diabetic patients (A Study

With NLY01 in Subjects With Type 2 Diabetes; ClinicalTrials.gov Identifier: NCT04159766), and patients with Parkinson's disease (A Clinical Study of NLY01 in Patient's With Early Parkinson's Disease; ClinicalTrials.gov Identifier: NCT04154072). However, there is still a need for non-immunogenic therapeutics for diabetes.

Therefore, it is an object of the invention to provide therapeutics with reduced immunogenicity and antigenicity that provide therapeutic benefit for metabolic disorders such as diabetes, without immune interference.

It is a further object of the present invention to provide therapeutics with increased in vivo half-life, stability, and extended duration of action.

It is a further object of the invention to provide deimmunized therapeutics and methods thereof that provide effective glucose control, with weight loss benefits and a favorable side effect profile.

### SUMMARY OF THE INVENTION

It has been established that glucagon-like peptide-1 (GLP-1) receptor agonist peptides where one or more major sites of immunogenicity are eliminated retain their bioactivity towards a GLP-1 receptor and is suitable for less frequent administration (e.g., once monthly) that provide comparable or better therapeutic efficacy compared to currently marketed GLP-1 RAs.

GLP-1R agonists or analogs thereof with reduced immunogenicity that provide comparable or better therapeutic efficacy in vivo are described. Typically, the GLP-1R agonists or analogs thereof have the amino acid sequence of
HGX₃GX₅X₆X₇X₈X₉X₁₀X₁₁X₁₂X₁₃X₁₄X₁₅X₁₆X₁₇X₁₈X₁₉X₂₀X₂₁X₂₂X₂₃X₂₄WX₂₆
X₂₇NGGPX₃₂X₃₃GX₃₅PPPX₃₉C (SEQ ID NO: 1), where Xₙ is each independently selected from the amino acids for the specific positions:
   X₃ is E, D, K, or Q;
   X₅ is S or T;
   X₆ is For Y; X₇ is S or T;
   X₈ is S, or A, D, E, G, K, N, Q, or T;
   X₉ is D, P, or E;
   X₁₀ is L, F, I, M, or V;
   X₁₁ is S, A, D, E, G, K, N, Q, or T;
   X₁₂ is K, E, Q, or R;
   X₁₃ is Q, E, K, or R;
   X₁₄ is M, R, L, or V;
   X₁₅ is E, D, K, or Q;
   X₁₆ is E, D, K, or Q;
   X₁₇ is E, D, K, or Q;
   X₁₈ is A, G, S, T, or V;
   X₁₉ is A, I, L, M, T, or V;
   X₂₀ is R, K, or Q;
   X₂₁ is L, F, I, M, or V;
   X₂₂ is For Y;
   X₂₃ is I, G, L, F, M, or V;
   X₂₄ is E, D, K, or Q;
   X₂₆ is L, F, I, M, or V;
   X₂₇ is K, E, Q, or R;
   X₃₂ is S, A, D, E, G, K, N, Q, or T;
   X₃₃ is S, A, D, E, G, K, N, Q, or T;
   X₃₅ is A, G, S, T, or V; and
   X₃₉ is S, A, D, E, G, K, N, Q, or T.

In some embodiments, the GLP-1R agonists or analogs thereof have the amino acid sequence of HGEGTFTSX₉LSKQMEEEAX₁₉RLFX₂₃EWLKNGGPSSGX₃₅PPPSC (SEQ ID NO:2). In preferred embodiments, X₁₉ is not a valine. In one embodiment, X₁₉ is A or T. In another embodiment, X₂₃ is G. In a further embodiment, X₉ is P and X₂₃ is G. Preferably, the GLP-1R agonists or analogs thereof have the amino acid sequence of any one of SEQ ID NOs: 2-7. In one embodiment, the GLP-1R agonists or analogs thereof have the amino acid sequence of SEQ ID NO: 5. Generally, the GLP-1RA peptides or analogs have a substantially lower immunogenicity in a mammalian host, preferably, human host, relative to immunogenicity of SEQ ID NO: 3. Typically, the peptide or analog is conjugated to one or more polyethylene glycol (PEG) moieties, for example, multiple-branched PEGs, having a molecular weight of between 20 kDa and 250 kDa. In preferred embodiments, the multiple-branched polyethylene glycol is a 3-arm branched PEGs having a molecular weight of between 20 kDa and 250 kDa, for example having one of the following structures:

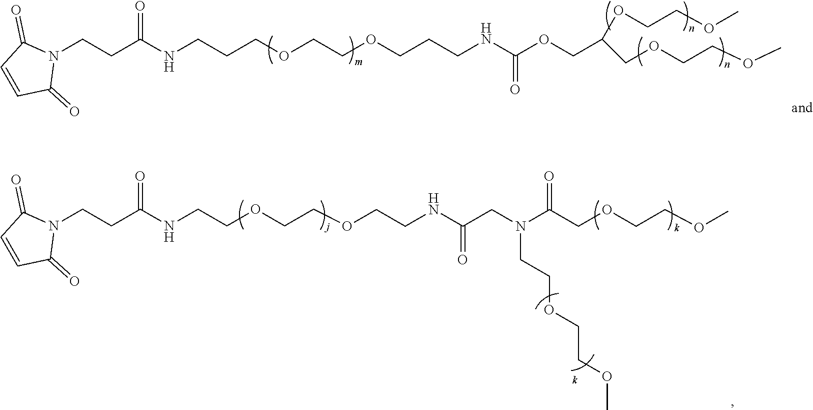

where m, n, j, k each is an integer independently between about 100 and 5,000.

In one embodiment, the multiple-branched polyethylene glycol is a 3-arm branched PEGs having a molecular weight of about 50 kDa. Exemplary GLP-1RA peptide or analog conjugated to one or more polyethylene glycol (PEG) moieties include any one of SEQ ID NOs: 7-13. In further preferred embodiments, the GLP-1RA peptide or analog has the amino acid sequence of SEQ ID NO: 5, and conjugated via C-terminal cysteine residue a 3-arm branched PEGs having one of the following structures:

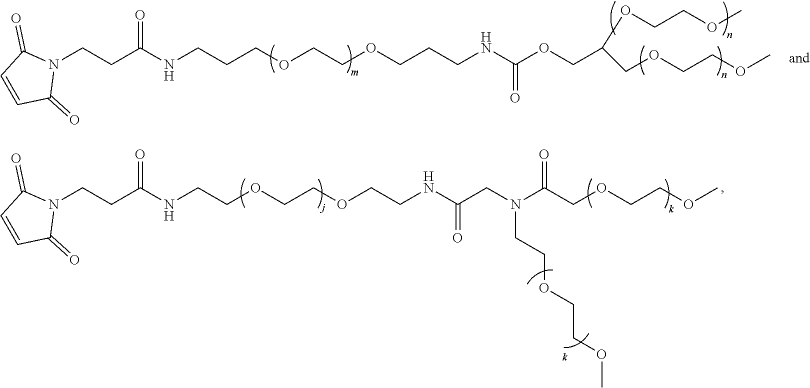

where m, n, j, k each is an integer independently between about 100 and 5,000, and the 3-arm branched PEG has a molecular weight of about 50 kDa.

Pharmaceutical formulations of the GLP-1 receptor agonist peptide or analog thereof, and methods of use thereof are also described.

Method of treating one or more diseases selected from the group consisting of obesity, diabetes, and non-alcoholic fatty liver disease in a subject in need thereof are provided. The methods include administering an effective amount of the pharmaceutical formulation of the GLP-1RA peptide or analog thereof to treat or alleviate one or more symptom of the one or more diseases. In preferred embodiments, the pharmaceutical formulation is administered in an amount effective to induce weight loss, reduce the body fat, reduce food intake, improve glucose homeostasis, or combinations thereof, in a normal or obese patient.

Typically, the pharmaceutical formulation is administered via enteral administration and parenteral administration, for example, oral administration or subcutaneous administration. In some embodiments, the pharmaceutical formulation is administered in a form of pills, capsules, tablets, liquids, and suspensions. In some embodiments, the pharmaceutical formulation is administered at an interval of once a month, once every two weeks, once a week, once every three days, once every two days, once daily, or twice daily. In preferred embodiments, the pharmaceutical formulation is administered once a month. In other embodiments, the pharmaceutical formulation is administered the subject once a week for up to 6 months, or for a duration of one to ten years, inclusive. In some embodiments, the pharmaceutical formulation is administered to a human subject at a dose of between 0.001 mg/kg body weight of the subject and 10 mg/kg body weight of the subject, inclusive. In preferred embodiments, the pharmaceutical formulation is administered to a human subject at a dose of between 0.01 mg/kg body weight of the subject and 1 mg/kg body weight of the subject, inclusive. In further embodiments, the pharmaceutical formulation is administered to a human subject at a dose of between 1.0 mg and 100 mg, inclusive.

Preferably, the pharmaceutical formulation provides a therapeutically effective concentration of the GLP-1RA peptide or analog in the serum of the subject throughout the treatment once a steady state is established after administration. In other embodiments, the therapeutically effective concentration of the GLP-1RA peptide or analog in the serum of the subject during the treatment does not fall below pharmacologically active levels at any given time point during the treatment. In preferred embodiments, the pharmaceutical formulation results in minimal side effect during the treatment.

In further preferred embodiments, the pharmaceutical formulation results in less incidence of nausea and vomiting and better gastrointestinal (GI) tolerance compared to albiglutide when both are administered once monthly. In one embodiment, the pharmaceutical formulation provides a minimal serum concentration of about 27 nM throughout the treatment once a steady state is established after administration.

### BRIEF DESCRIPTION OF THE DRAWINGS

FIGS. 1A-1B are molecular structures of exemplary 3-arm branched 50 kD PEG variants including NPEG (FIG. 1A) and JPEG (FIG. 1B), respectively.

FIGS. 2A-2D show the results of immunogenicity prediction analysis on three peptides Exenatide-Cys (FIG. 2A), Exenatide (V19T)-Cys (FIG. 2B), and Exenatide (V19A)-Cys (FIG. 2C). Each row in the table represents the position of the P1 anchor residue of a 9 mer peptide which is scored for MHC class II binding to all 46 HAL-DR, DP and DQ alleles in the right-hand side column (‘Position Risk Score’). A ‘Position Risk Score’ of 1-2, 3-5, 6+ is highlighted in light, medium or dark grey, respectively. ‘Total Score’ corresponds to the sum of all ‘Position Risk Scores’ for the whole sequence and ‘Hotspot Max’ to the single highest ‘Position Risk Score’. FIG. 2D depicts additional possible amino acid substitutions at position 9, 23 and 35 predicted to have reduced MHC Class II binding.

FIG. 3 is a schematic depicting the method of ipGTT tests conducted at 24, 48, and 72 hours after a single subcutaneous dose.

FIG. 4 is a graph of comparison of ipGTT in normal mice following treatment with NLY01 with two different PEG sources (Exenatide-Cys-NPEG50K and Exenatide-Cys-JPEG50K) and the V19 deimmunized pegylated exenatide variants (Exenatide (V19T)-Cys-JPEG50K and Exenatide (V19A)-Cys-JPEG50), showing blood glucose level (mg/dL) over Time (0-26 h) for each of G1-Vehicle (saline; •), G2-Ozempic (semaglutide; ▪), G3-Ozempic (semaglutide; ▴), G4-Exenatide-Cys-NPEG50K (NLY01; ▾), G5-Exenatide-Cys-JPEG50K (♦), G6-Exenatide (V19T)-Cys-JPEG50K (●), and G7-Exenatide (V19A)-Cys-JPEG50K (▪), respectively.

FIG. 5 is a graph of comparison of blood glucose levels in db/db mice following treatment with NLY01 with two different PEG sources (Exenatide-Cys-NPEG50K and Exenatide-Cys-JPEG50K) and the V19 deimmunized pegylated exenatide variants, Exenatide (V19T)-Cys-JPEG50K and Exenatide (V19A)-Cys-JPEG50, showing blood glucose level (mg/dL) over Time (0-96 h) for each of G1-Vehicle (•), G2-(NLY01, SC, 15 nmol/kg; ▪), G-3 (Exenatide-Cys-JPEG50K, SC, 15 nmol/kg; ▴), G4-(Exenatide (V19T)-Cys-JPEG50K, SC, 15 nmol/kg; ▾), G5-Exenatide (V19A)-Cys-JPEG50K, SC, 15 nmol/kg; ♦), and G6-Ozempic (semaglutide), SC, 15 nmol/kg; (●), respectively.

FIGS. 6A-6D are graphs of the effects of repeated administration of Exenatide (V19A)-Cys-JPEG50 (NLY12) on fasting blood glucose and HbA1c in a diabetic mice model, showing blood glucose (0-600 mg/dL) over Time (0-28 days) for each of G1 (Vehicle; •), G2 (NLY012, SC, 1 mg/kg; ▪), G3 (NLY012, SC, 3 mg/kg; ▴) (FIG. 6A); blood glucose % change from baseline over Time (0-28 days) for each of G1 (Vehicle; •), G2 (NLY012, SC, 1 mg/kg; ▪), G3 (NLY012, SC, 3 mg/kg; ▴) (FIG. 6B); HbA1c (4-10%) over Time (0-28 days) for each of G1 (Vehicle; •), G2 (NLY012, SC, 1 mg/kg; ▪), G3 (NLY012, SC, 3 mg/kg; ▴) (FIG. 6C); and HbA1c % change from baseline at 4 weeks for each of G1 (Vehicle; •), G2 (NLY012, SC, 1 mg/kg; ▪), G3 (NLY012, SC, 3 mg/kg; ▴) (FIG. 6D).

FIGS. 7A-7H are graphs of effects of repeated administration of Exenatide (V19A)-Cys-JPEG50 (NLY12) on intra-peritoneal glucose tolerance test (ipGTT) in diabetic mice model, showing: for Day 1 blood glucose (0-300 mg/dL) over Time (0-120 mins) for each of G1 (Vehicle; •), G2 (NLY012, SC, 1 mg/kg, SC, BW; ▪), G3 (NLY012, SC, 3 mg/kg, SC, BW; ▴) (FIG. 7A); AUC (0-20,000 mg/dL-min) for each of G1 (Vehicle; •), G2 (NLY012, SC, 1 mg/kg, SC, BW; ▪), G3 (NLY012, SC, 3 mg/kg, SC, BW; ▴) (FIG. 7B); for ipGTT Day 1 blood glucose (0-300 mg/dL) over Time (0-120 mins) for each of G1 (Vehicle; •), G2 (NLY012, SC, 1 mg/kg, SC, BW; ▪), G3 (NLY012, SC, 3 mg/kg, SC, BW; ▴) (FIG. 7C); AUC (0-20,000 mg/dL-min) for each of G1 (Vehicle; •), G2 (NLY012, SC, 1 mg/kg, SC, BW; ▪), G3 (NLY012, SC, 3 mg/kg, SC, BW; ▴) (FIG. 7D); and for ipGTT Day 27 blood glucose (0-300 mg/dL) over Time (0-120 mins) for each of G1 (Vehicle; •), G2 (NLY012, SC, 1 mg/kg, SC, BW; ▪), G3 (NLY012, SC, 3 mg/kg, SC, BW; ▴) (FIG. 7E); AUC (0-20,000 mg/dL-min) for each of G1 (Vehicle; •), G2 (NLY012, SC, 1 mg/kg, SC, BW; ▪), G3 (NLY012, SC, 3 mg/kg, SC, BW; ▴) (FIG. 7F); for ipGTT Day 27 blood glucose (0-300 mg/dL) over Time (0-120 mins) for each of G1 (Vehicle; •), G2 (NLY012, SC, 1 mg/kg, SC, BW; ▪), G3 (NLY012, SC, 3 mg/kg, SC, BW; ▴) (FIG. 7G); and AUC (0-20,000 mg/dL-min) for each of G1 (Vehicle; •), G2 (NLY012, SC, 1 mg/kg, SC, BW; ▪), G3 (NLY012, SC, 3 mg/kg, SC, BW; ▴) (FIG. 7H).

FIG. 8 is a graph of the pharmacokinetic profile of the comparison of NLY01 with different PEG sources (Exenatide-Cys-NPEG50K and Exenatide-Cys-JPEG50K) and the V19 deimmunized pegylated exenatide variants, Exenatide (V19T)-Cys-JPEG50K and Exenatide (V19A)-Cys-JPEG50 in non-human primate, showing conc. in serum (1-1000 μg/ml) over

Time (0-504 hours) for each of Exenatide-Cys-NPEG50K, NLY01 (•), Exenatide-Cys-JPEG50K (▴), Exenatide (V19T)-Cys-JPEG50K (▪), and Exenatide (V19A)-Cys-JPEG50K (▾), respectively.

FIG. 9 is a graph of the NLY12, NLY01 and semaglutide in a GLP-1R cAMP potency assay, showing c-AMP release (% of Maximal Response) over Concentration (Log nM) for each of NLY01 (▪); NLY12 (●); and Semaglutide (□), respectively.

FIG. 10 is a graph of the Food intake of Obese/T2D NHPs treated with NLY12, showing Total Energy Intake (Kcal) over Study Days (−28 to 63) for each of Vehicle (♦) NLY12 Weekly (▪); and NLY12 biweekly (▴), respectively.

FIG. 11 is a graph of the Change in Body Weight of Obese/T2D NHPs treated with NLY12, showing % change in body weight over Study Days (−10 to 63) for each of Vehicle (♦), NLY12 Weekly (▪) and NLY12 biweekly (▴) respectively.

FIG. 12 is a graph of the Change in Fasting Glucose in Obese/T2D NHPs treated with NLY12, showing % change in glucose over Study Days (0 to 65) for each of Vehicle (♦), NLY12 Weekly (▪); and NLY12 biweekly (▴), respectively.

FIG. 13 is a graph of the Change in HbA1c in Obese/T2D NHPs treated with NLY12, showing % change in HbA1c over Study Days (0 to 50) for each of Vehicle (♦), NLY12 Weekly (▪); and NLY12 biweekly (▴), respectively.

FIG. 14 is a graph of the Serum NLY12 Concentration in Obese/T2D NHPs treated with NLY12, showing conc. in serum (1-100 μg/ml) over Time (0-1176 hours) for each of Group 2 (NLY12, weekly; •), and Group 3 (NLY12, biweekly; ▴), respectively.

FIGS. 15A-15C depict the pharmacokinetics of NLY12 following Titration and Monthly Dosing, showing a graph of NLY12 (ng/ml) over time (weeks 0-40) for each of NLY12 Monthly (−) and Semaglutide (−−), with 75 nM Wegovy, as well as 65 nM and 27 nM Ozempic indicated (FIG. 15A); and Tables showing NLY12 Steady State at 40 mg Monthly (FIG. 15B) and Therapeutic Levels of Semaglutide (FIG. 15C), respectively.

### DETAILED DESCRIPTION OF THE INVENTION

### I. Definitions

The term “immunogenicity”, in the context of a polypeptide, conjugate, or a composition refers to a polypeptide, a conjugate, or a composition that can induce an immune response and is therefore antigenic. By “immune response” means any reaction by the immune system. These reactions include the alteration in the activity of an organism's immune system in response to a polypeptide, polynucleotide, conjugate, or compositions thereof, and can involve, for example, antibody production, induction of cell-mediated immunity, complement activation.

The phrase “substantially lower immunogenicity” specifies a reduction or inhibition of immunogenicity score of greater than at least about 25%, at least about 20%, at least about 15%, at least about 12.5%, at least about 10%, or at least about 5%, relative to the immunogenicity of a corresponding polypeptide or a corresponding conjugate in a host predicted by the in silico modeling, or in immune cells ex vivo.

The term “corresponding polypeptide” refers to a polypeptide formed generally of similar sequence as a reference polypeptide, but without the modifications of the reference polypeptide.

The term “corresponding conjugate” refers to a conjugate formed generally of the same or similar sequence as a reference conjugate, but without the modifications of the reference conjugate.

The term “polypeptides” includes proteins and fragments thereof. Polypeptides are amino acid residue sequences. Those sequences are written left to right in the direction from the amino to the carboxy terminus. In accordance with standard nomenclature, amino acid residue sequences are denominated by either a three letter or a single letter code as indicated as follows: Alanine (Ala, A), Arginine (Arg, R), Asparagine (Asn, N), Aspartic Acid (Asp, D), Cysteine (Cys, C), Glutamine (Gln, Q), Glutamic Acid (Glu, E), Glycine (Gly, G), Histidine (His, H), Isoleucine (Ile, I), Leucine (Leu, L), Lysine (Lys, K), Methionine (Met, M), Phenylalanine (Phe, F), Proline (Pro, P), Serine (Ser, S), Threonine (Thr, T), Tryptophan (Trp, W), Tyrosine (Tyr, Y), and Valine (Val, V).

The term “variant” refers to a polypeptide or polynucleotide that differs from a reference polypeptide or polynucleotide, but retains essential properties. A typical variant of a polypeptide differs in amino acid sequence from another, reference polypeptide. Generally, differences are limited so that the sequences of the reference polypeptide and the variant are closely similar overall and, in many regions, identical. A variant and reference polypeptide may differ in amino acid sequence by one or more modifications (e.g., substitutions, additions, and/or deletions). A substituted or inserted amino acid residue may or may not be one encoded by the genetic code. A variant of a polypeptide may be naturally occurring such as an allelic variant, or it may be a variant that is not known to occur naturally.

Modifications and changes can be made in the structure of the polypeptides and still obtain a molecule having similar characteristics as the polypeptide (e.g., a conservative amino acid substitution). For example, certain amino acids can be substituted for other amino acids in a sequence without appreciable loss of activity. Because it is the interactive capacity and nature of a polypeptide that defines that polypeptide's biological functional activity, certain amino acid sequence substitutions can be made in a polypeptide sequence and nevertheless obtain a polypeptide with like properties.

In making such changes, the hydropathic index of amino acids can be considered. The importance of the hydropathic amino acid index in conferring interactive biologic function on a polypeptide is generally understood in the art. It is known that certain amino acids can be substituted for other amino acids having a similar hydropathic index or score and still result in a polypeptide with similar biological activity. Each amino acid has been assigned a hydropathic index on the basis of its hydrophobicity and charge characteristics. Those indices are: isoleucine (+4.5); valine (+4.2); leucine (+3.8); phenylalanine (+2.8); cysteine/cysteine (+2.5); methionine (+1.9); alanine (+1.8); glycine (−0.4); threonine (−0.7); serine (−0.8); tryptophan (−0.9); tyrosine (−1.3); proline (−1.6); histidine (−3.2); glutamate (−3.5); glutamine (−3.5); aspartate (−3.5); asparagine (−3.5); lysine (−3.9); and arginine (−4.5).

It is believed that the relative hydropathic character of the amino acid determines the secondary structure of the resultant polypeptide, which in turn defines the interaction of the polypeptide with other molecules, such as enzymes, substrates, receptors, antibodies, and antigens. It is known in the art that an amino acid can be substituted by another amino acid having a similar hydropathic index and still obtain a functionally equivalent polypeptide. In such changes, the substitution of amino acids whose hydropathic indices are within ±2 is preferred, those within ±1 are particularly preferred, and those within ±0.5 are even more particularly preferred.

Substitution of like amino acids can also be made on the basis of hydrophilicity. The following hydrophilicity values have been assigned to amino acid residues: arginine (+3.0); lysine (+3.0); aspartate (+3.0±1); glutamate (+3.0±1); serine (+0.3); asparagine (+0.2); glutamnine (+0.2); glycine (0); proline (−0.5+1); threonine (−0.4); alanine (−0.5); histidine (−0.5); cysteine (−1.0); methionine (−1.3); valine (−1.5); leucine (−1.8); isoleucine (−1.8); tyrosine (−2.3); phenylalanine (−2.5); tryptophan (−3.4). It is understood that an amino acid can be substituted for another having a similar hydrophilicity value and still obtain a biologically equivalent, and in particular, an immunologically equivalent polypeptide. In such changes, the substitution of amino acids whose hydrophilicity values are within ±2 is preferred, those within ±1 are particularly preferred, and those within ±0.5 are even more particularly preferred.

As outlined above, amino acid substitutions are generally based on the relative similarity of the amino acid side-chain substituents, for example, their hydrophobicity, hydrophilicity, charge, and size. In particular, embodiments of the polypeptides can include variants having about 50%, 60%, 70%, 80%, 90%, and 95% sequence identity to the polypeptide of interest.

The term “solubility” refers to maximum concentration of the polypeptide or conjugate in a physiological buffer, at physiological pH and room temperature, at which the polypeptide or conjugate does not substantially aggregate. Solubility and/or aggregation may be detected by chromatography, gel electrophoresis, or melting/aggregations analyses.

The term “half-life in vivo” generally refers to a half of the maximum time the polypeptide or conjugate is retained in circulation in vivo. The half-life in vivo may be measured by activity assays specific to the polypeptide or conjugate after obtaining plasma samples at different time intervals from a subject administered the polypeptide or conjugate. The half-life in vivo may be measured by detection assays detecting the presence of the polypeptide or conjugate in plasma samples obtained at different time intervals from a subject administered the polypeptide or conjugate.

The term “host”, “subject” or “patient” refers to any individual who is the target of administration.

The term “effective amount” or “therapeutically effective amount” means a dosage sufficient to treat, inhibit, or alleviate one or more symptoms of a disease state being treated or to otherwise provide a desired pharmacologic and/or physiologic effect. The precise dosage will vary according to a variety of factors such as subject-dependent variables (e.g., age, immune system health, etc.), the disease or condition being treated, the severity of the disease or disorder, and the treatment being administered. The effect of the effective amount can be relative to a control. Such controls are known in the art and discussed herein, and can be, for example, the condition of the subject prior to or in the absence of administration of the drug, or drug combination, or in the case of drug combinations, the effect of the combination can be compared to the effect of administration of only one of the drugs. One of ordinary skill in the art may empirically determine the effective amount of a particular compound without necessitating undue experimentation. In some embodiments, the term “effective amount” refers to an amount of a GLP-1RA peptide or analogs thereof to reduce or diminish the risk of developing type 2 diabetes and obesity or to reduce or diminish one or more symptoms of type 2 diabetes and obesity, such as high fasting blood glucose levels (e.g., 126 mg/dL (7 mmol/L) or higher). Additional desired results also include reducing HbA1c level, and body weight; and increasing glucose tolerance.

The term “combination therapy” refers to treatment of a disease or symptom thereof, or a method for achieving a desired physiological change, including administering an effective amount of two or more chemical agents or components to treat the disease or symptom thereof, or to produce the physiological change, wherein the chemical agents or components are administered together, such as part of the same composition, or administered separately and independently at the same time or at different times (i.e., administration of each agent or component is separated by a finite period of time from each other).

The term “substantially” refers to comparative measurement of at least about 25%, at least about 20%, at least about 15%, at least about 12.5%, at least about 10%, or at least about 5%, relative to a control.

The term “therapeutic agent” refers to an agent that can be administered to treat one or more symptoms of a disease or disorder.

The term “pharmaceutically acceptable salt”, as used herein, refers to derivatives of the compounds defined herein, wherein the parent compound is modified by making acid or base salts thereof. Example of pharmaceutically acceptable salts include, but are not limited to, mineral or organic acid salts of basic residues such as amines; and alkali or organic salts of acidic residues such as carboxylic acids. Pharmaceutically acceptable salts include the conventional non-toxic salts or the quaternary ammonium salts of the parent compound formed, for example, from non-toxic inorganic or organic acids. Such conventional non-toxic salts include those derived from inorganic acids such as hydrochloric, hydrobromic, sulfuric, sulfamic, phosphoric, and nitric acids; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, tolunesulfonic, naphthalenesulfonic, methanesulfonic, ethane disulfonic, oxalic, and isethionic salts.

The phrase “pharmaceutically acceptable” or “biocompatible” refers to compositions, polymers, and other materials and/or dosage forms which are, within the scope of sound medical judgment, suitable for use in contact with the tissues of human beings and animals without excessive toxicity, irritation, allergic response, or other problem or complication, commensurate with a reasonable benefit/risk ratio. The phrase “pharmaceutically acceptable carrier” refers to pharmaceutically acceptable materials, compositions, or vehicles, such as a liquid or solid filler, diluent, solvent, or encapsulating material involved in carrying or transporting any subject composition, from one organ, or portion of the body, to another organ, or portion of the body. Each carrier must be “acceptable” in the sense of being compatible with the other ingredients of a subject composition and not injurious to the patient.

The terms “inhibit” or “reduce” in the context of inhibition, mean to reduce or decrease in activity and quantity. This can be a complete inhibition or reduction in activity or quantity, or a partial inhibition or reduction. Inhibition or reduction can be compared to a control or to a standard level. Inhibition can be 5, 10, 25, 50, 75, 80, 85, 90, 95, 99, or 100%. For example, long-lasting GLP-1r agonists may inhibit or reduce the activity and/or quantity of activated microglia by about 10%, 20%, 30%, 40%, 50%, 75%, 85%, 90%, 95%, or 99% from the activity and/or quantity of the same cells in equivalent tissues of subjects that did not receive or were not treated with long-lasting GLP-1r agonists. In some embodiments, the inhibition and reduction are compared at mRNAs, proteins, cells, tissues, and organs levels.

The term “treating” or “treatment” refers to amelioration, alleviation or reduction of one or more symptoms of a disease, disorder, or condition in an person who may be predisposed to the disease, disorder and/or condition but has not yet been diagnosed as having it; reducing disease symptoms, inhibiting the disease, disorder or condition, e.g., impeding its progress; and relieving the disease, disorder, or condition, e.g., causing regression of the disease, disorder and/or condition. Treating the disease or condition includes ameliorating at least one symptom of the particular disease or condition, even if the underlying pathophysiology is not affected, such as treating the pain of a subject by administration of an analgesic agent even though such agent does not treat the cause of the pain. Desirable effects of treatment include decreasing the rate of disease progression, ameliorating, or palliating the disease state, and remission or improved prognosis. For example, an individual is successfully “treated” if one or more symptoms associated with diseases/disorders are mitigated or eliminated, including, but not limited to, reducing and/or inhibiting elevations of fasting blood glucose levels, HbA1c level, and body weight. The term “ameliorate” refers to a decrease, suppression, attenuation, diminish, arrest, or stabilization of the development or progression of a disease.

The terms “protein” or “polypeptide” or “peptide” refer to any chain of more than two natural or unnatural amino acids, regardless of post-translational modification (e.g., glycosylation or phosphorylation), constituting all or part of a naturally occurring or non-naturally occurring polypeptide or peptide.

The term “PEGylation” refers to a process of both covalent and non-covalent attachment or amalgamation of polyethylene glycol (PEG) polymer chains to molecules and macrostructures, such as a drug, therapeutic protein, or vesicle.

Use of the term “about” is intended to describe values either above or below the stated value in a range of approx. +/−10%; in other embodiments the values may range in value either above or below the stated value in a range of approx. +/−5%.

### II. Compositions

### A. GLP-1RA Peptides

Compositions including isolated peptides having activities to a glucagon-like peptide-1 (GLP-1) receptor are provided. Long-acting glucagon like peptide 1 receptor agonists (long-acting GLP-1R agonists), such as exenatide and PEGylated exenatide, reduce and inhibit pathological processes such as microglial activation. In preferred embodiments, long-acting GLP-1R agonists are exenatide with one or more amino acid substitutions and one or more modifications (e.g., PEGylation) to reduce immunogenicity and antigenicity, compared to a control (e.g., exenatide without the substitutions and/or modifications). These de-immunized GLP-1R agonists are stable, soluble, has an increased half-life in vivo, and reduced immunogenicity. In preferred embodiments, the de-immunized GLP-1R agonists have similar in vivo efficacy compared to exenatide but better suited for long term usage based on their reduced immunogenicity.

### 1. Deimmunized GLP-1RA Peptides

In some embodiments, the GLP-1RA peptides are deimmunized exenatide peptides, i.e., exenatide peptide variants with one or more major sites of immunogenicity eliminated from the peptide. Methods to assess and predict immunogenicity potential for drug candidate in development were used, such as Rosenberg, et al Pharm. Pharmacol. 70(5), 584-594 (2018). For example, computer algorithms can be used to predict MHC-II epitope and peptide-HLA II binding affinities. Peptide-HLA II binding affinities may be estimated with in vitro assays. The immunogenicity of a full therapeutic molecule, including protein/peptide and modifications, can be assessed by in vitro by measuring T-cell proliferation after exposing full molecule to ex vivo cultured lymphocytes from human donors providing a readout for immunogenic potential.

Thus, in some embodiments, the GLP-1RA peptides are exenatide peptides with one or more amino acid substitution, addition, or deletion.

In some embodiments, the GLP-1RA peptides have the amino acid sequence of the following Polypeptide Formula (I):
HGX₃GX₅X₆X₇X₈X₉X₁₀X₁₁X₁₂X₁₃X₁₄X₁₅X₁₆X₁₇ X₁₈X₁₉X₂₀X₂₁X₂₂X₂₃X₂₄WX₂₆X₂₇NGGPX₃₂X₃₃GX₃₅PPPX₃₉C (SEQ ID NO: 1), where Xₙ indicates any one of the 20 amino acid or derivatives thereof.

In some embodiments, X is each independently selected from the following defined amino acids for the specific positions where X₃ is E, D, K, or Q; X₅ is S or T; X₆ is For Y; X₇ is S or T; X₈ is S, or A, D, E, G, K, N, Q, or T; X₉ is D, P, K, or E; X₁₀ is L, F, I, M, or V; X₁₁ is S, A, D, E, G, K, N, Q, or T; X₁₂ is K, E, Q, or R; X₁₃ is Q, E, K, or R; X₁₄ is M, R, L, or V; X₁₅ is E, D, K, or Q; X₁₆ is E, D, K, or Q; X₁₇ is E, D, K, or Q; X₁₈ is A, G, S, T, or V; X₁₉ is A, I, L, M, T, or V; X₂₀ is R, K, or Q; X₂₁ is L, F, I, M, or V; X₂₂ is For Y; X₂₃ is I, G, L, F, M, or V; X₂₄ is E, D, K, or Q; X₂₆ is L, F, I, M, or V; X₂₇ is K, E, Q, or R; X₃₂ is S, A, D, E, G, K, N, Q, or T; X₃₃ is S, A, D, E, G, K, N, Q, or T; X₃₅ is A, G, S, T, or V; and X₃₉ is S, A, D, E, G, K, N, Q, or T.

In some embodiments, X₁₉ is A, I, L, M, or T. In preferred embodiments, X₁₉ is not V. In preferred embodiments, X₁₉ is A or T. In one embodiment, X₁₉ is A. In another embodiment, X₁₉ is T.

In some embodiments, X₉ is D, P, K or R. In some embodiments, X₂₃ is I or G. In some embodiments, X₃₅ is A, T, N, H, R, or G.

In some embodiments, the GLP-1RA peptides have the amino acid sequence of the following Polypeptide Formula (II): HGEGTFTSX₉LSKQMEEEAX₁₉RLFX₂₃EWLKNGGPSSGX₃₅PPPSC (SEQ ID NO:2), where X₉ is D, P, or K; X₁₉ is A, T, or V; X₂₃ is I or G; X₃₅ is A, G, H, T, R, or N.

In preferred embodiments, X₁₉ is not V in Polypeptide Formula (II). In preferred embodiments, X₁₉ is A or T. In one embodiment, X₁₉ is A. In another embodiment, X₁₉ is T.

In one embodiment, the GLP-1RA peptide has the amino acid sequence of SEQ ID NO: 3 as shown below:

(SEQ ID NO: 3)
HGEGTFTSDLSKQMEEEAVRLFIEWLKNGGPSSGAPPPSC.

In another preferred embodiment, X₁₉ is T in Polypeptide Formula I and the GLP-1RA peptide has the amino acid sequence of SEQ ID NO: 4 as shown below:

(SEQ ID NO: 4)
HGEGTFTSDLSKQMEEEATRLFIEWLKNGGPSSGAPPPSC.

In a preferred embodiment, X₁₉ is A in Polypeptide Formula I and the GLP-1RA peptide has the amino acid sequence of SEQ ID NO: 5 as shown below:

(SEQ ID NO: 5)
HGEGTFTSDLSKQMEEEAARLFIEWLKNGGPSSGAPPPSC.

In a further embodiment, X₂₃ is G in Polypeptide Formula I and the GLP-1RA peptide has the amino acid sequence of SEQ ID NO: 6 as shown below:

(SEQ ID NO: 6)
HGEGTFTSDLSKQMEEEAVRLFGEWLKNGGPSSGAPPPSC.

In a further embodiment, X₉ is P and X₂₃ is G in Polypeptide Formula I, and the GLP-1RA peptide has the amino acid sequence of SEQ ID NO: 7 as shown below:

(SEQ ID NO: 7)
HGEGTFTSPLSKQMEEEAVRLFGEWLKNGGPSSGAPPPSC.

Cysteine (C40) at C-terminus of Polypeptide Formula (I) allows further modifications such as pegylation. Thus, in preferred embodiments, Cysteine (C40) of any one of SEQ ID NOs: 1-7 is pegylated.

### a. Biological Activity

Typically, the de-immunized GLP-1R agonists maintain bioactivity in vivo. As shown in Example 2, for example, the de-immunized GLP-1R agonists provided effective reductions in fasting blood glucose, HbA1c level, and improve glucose tolerance in diabetic mice. Further, the de-immunized GLP-1R agonists were effective in reducing body weight, fasting blood glucose, HbA1c level, and improving glucose tolerance in diabetic monkey.

In some embodiments, the de-immunized GLP-1R agonists show low immunogenicity T-cell proliferation assay compared to equivalent exenatide. In preferred embodiments, the GLP-1RA peptide analogs have EC₅₀ comparable to NLY01 (Exenatide-Cys-NPEG50K and Exenatide-Cys-JPEG50K), in cell-based GLP-1R cAMP assays.

In some embodiments, GLP-1RA peptide analogs have in vivo efficacy comparable to NLY01 (Exenatide-Cys-NPEG50K and Exenatide-Cys-JPEG50K) or other positive controls such as semaglutide in vivo studies, for example, using mouse models. In some embodiments, GLP-1RA peptide analogs have in vivo efficacy (e.g., reductions in blood glucose, HbA1c and ipGTT) comparable to NLY01 (Exenatide-Cys-NPEG50K) or Exenatide-Cys-JPEG50K or other positive controls such as semaglutide.

In preferred embodiments, the de-immunized GLP-1R agonists do not result differences in treatment-related effects on body weight, hematology, coagulation, or serum chemistry parameters. In some embodiments, the GLP-1RA peptide analogs (similarly modified as control, e.g., JPEG50K) have pharmacokinetic parameters (Cmax, AUC, T_{1/2}) comparable to NLY01 (Exenatide-Cys-NPEG50K) or Exenatide-Cys-JPEG50K.

### b. Low Immunogenicity

Typically, the de-immunized GLP-1R agonists have a lower immunogenicity in a host when compared to the immunogenicity of exenatide in the same host. Generally, the host is a mammal, preferably, human.

The lower immunogenicity may be a lower immunogenicity score for the de-immunized GLP-1R agonists when compared to that of exenatide. An exemplary in silico prediction of T cell epitopes such as MHC Class binding in GLP-1RAs immunogenicity hot spot is iTope-AI, which predicts peptide binding specificities across 46 major HLA-DR, DP and DQ Isotypes.

Typically, the de-immunized GLP-1R agonists produce minimal or lower levels of T cell activation markers in a host when compared to the level of T cell activation makers produced by exenatide in the same host, as detected by T cell assay or an equivalent assay. In preferred embodiments, comparison is carried out when the de-immunized GLP-1R agonists and exenatide are similarly modified, for example, both with conjugation via C-terminal cysteine to a 3-arm PEG of about 50 kDa.

In preferred embodiments, the de-immunized GLP-1R agonists typically have at least about 10%, 15%, 20%, 25%, 30%, 35%, 40%, 45%, or 50% lower total immunogenicity score when compared to exenatide or NLY01. For example, two peptide sequences of interest can be analyzed by predictive algorithm iTope-AI to identify peptides that bind to human MHC class II and/or share homology to known T cell epitopes. As described in Example 1, it was determined that SEQ ID NO:3, SEQ ID NO:4, and SEQ ID NO: 5 had a Total Score of 29, 19, and 27 respectively, with none of the peptide sequences matching a T cell epitope in T Cell Epitope Database (TCED™, Abzena database).

The currently available GLP-1R agonists such as exenatide (BYDUREON®) are immunogenic with 43%-49% of patients developing antibodies and 6% with an attenuated glycemic response when administered once weekly. Such immune response to GLP-1RA is associated with reduced effectiveness in some patients and can also be associated with injection site reactions or, in some cases, a systemic allergic response. Thus, the lower immunogenicity of de-immunized GLP-1R agonists provide a better and durable treatment for patients. In preferred embodiment, the de-immunized GLP-1R agonists maintain bioactivity while achieving an extended serum half-life of in humans, preferably more than 5 days, 6 days, 7 days, 8 days, 9 days, 10 days, 11 days, 12 days, 13 days, 14 days, or 15 days. In one embodiment, the extended serum half-life of in humans is more than 12 days.

### 2. Modifications to GLP-1RA Peptides

The direct use of native polypeptides as biopharmaceuticals is often limited by their very short systemic half-lives resulting from a rapid metabolism, enzymatic degradation, and, for smaller proteins and peptides, effective renal clearance. Modifications to exenatide such as semaglutide, liraglutide, and NLY01, have extended the half-life and pharmacokinetics of the active agent. Thus, further modifications are made to further improve the oral bioavailability, stability, and/or pharmacokinetics.

In some embodiments, the GLP-1RA peptides have the amino acid sequence of any one of SEQ ID NOs: 1-7. In preferred embodiments, the GLP-1RA peptides are GLP-1RA analogs modified with one or more polyethylene glycol moieties, optionally with one or more spacers, to achieve desired pharmacokinetics, stability, and bioavailability.

The choice of the suitable functional group for modifications is based on the type of available reactive group on the molecule that will be coupled to the PEG moieties. In preferred embodiments, the reactive amino acids are cysteine.

In preferred embodiments, one or more PEG moieties or derivative thereof, are conjugated to the amino acid sequence of any one of SEQ ID NOs: 1-7 via a C-terminal cysteine residue.

### a. Pegylation

In some embodiments, GLP-1RA peptides are modified with polyethylene glycol, polythyl imine, or derivatives thereof. In preferred embodiments, GLP-1RA peptides are modified by PEGylation via a C-terminal cysteine residue. Modifications can alter pharmacokinetics, pharmacodynamics, stability, and bioavailability.

Polyethylene glycol (PEG) is a polyether compound with many applications from industrial manufacturing to medicine. The structure of PEG is (note the repeated element in parentheses): H—(O—CH₂—CH₂)ₙ—OH. PEG is also known as polyethylene oxide (PEO) or polyoxyethylene (POE), depending on its molecular weight. PEG, PEO, or POE refers to an oligomer or polymer of ethylene oxide. The three names are chemically synonymous, but historically PEG is preferred in the biomedical field, whereas PEO is more prevalent in the field of polymer chemistry. Because different applications require different polymer chain lengths, PEG has tended to refer to oligomers and polymers with a molecular mass below 20,000 g/mol, PEO to polymers with a molecular mass above 20,000 g/mol, and POE to a polymer of any molecular mass. PEG and PEO are liquids or low-melting solids, depending on their molecular weights. PEGs are prepared by polymerization of ethylene oxide and are commercially available over a wide range of molecular weights from 300 g/mol to 10,000,000 g/mol. While PEG and PEO with different molecular weights find use in different applications, and have different physical properties (e.g., viscosity) due to chain length effects, their chemical properties are nearly identical. Different forms of PEG are also available, depending on the initiator used for the polymerization process—the most common initiator is a monofunctional methyl ether PEG, or methoxypoly(ethylene glycol), abbreviated mPEG. Lower-molecular-weight PEGs are also available as purer oligomers, referred to as monodisperse, uniform, or discrete. Very high purity PEG has recently been shown to be crystalline, allowing determination of a crystal structure by x-ray diffraction. Since purification and separation of pure oligomers is difficult, the price for this type of quality is often 10-10,00 fold that of polydisperse PEG.

PEGs are also available with different geometries. Branched PEGs have three to ten PEG chains emanating from a central core group. Star PEGs have 10 to 100 PEG chains emanating from a central core group. Comb PEGs have multiple PEG chains normally grafted onto a polymer backbone. The numbers that are often included in the names of PEGs indicate their average molecular weights (e.g., a PEG with n=9 would have an average molecular weight of approximately 400 Daltons and would be labeled PEG 400. Most PEGs include molecules with a distribution of molecular weights (i.e., they are polydisperse). The size distribution can be characterized statistically by its weight average molecular weight (Mw) and its number average molecular weight (Mn), the ratio of which is called the polydispersity index (Mw/Mn). MW and Mn can be measured by mass spectrometry.

In some embodiments, the polyethylene glycol or a derivative thereof is a linear type or a branched type, and for the branched type, preferably a dimeric type or a trimeric type may be used, and more preferably a trimeric type may be used. Specifically, the polyethylene glycol derivative is, for example, methoxypolyethylene glycol succinimidylpropionate, methoxypolyethylene glycol N-hydroxysuccinimide, methoxypolyethylene glycol propionaldehyde, methoxypolyethylene glycol maleimide, or multiple branched types of these derivatives. Preferably, the polyethylene glycol derivative is linear methoxypolyethylene glycol maleimide, branch type methoxypolyethylene glycol maleimide or trimeric methoxypolyethylene glycol maleimide, and more preferably is trimeric methoxypolyethylene glycol maleimide.

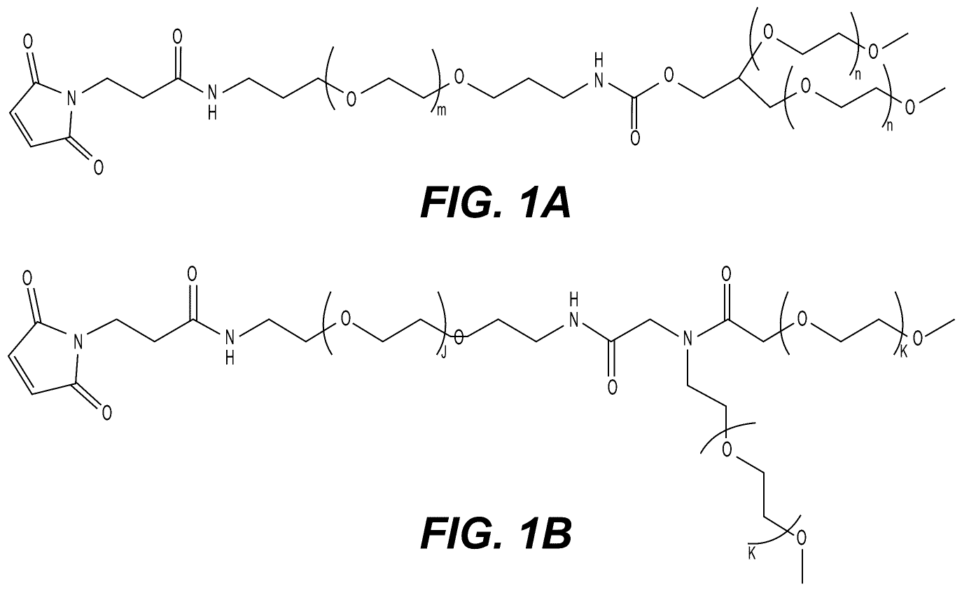

In some embodiments, the polyethylene glycol or a derivative thereof is at least 1,000 Daltons. In some embodiments, the polyethylene glycol or a derivative thereof is within the range of 1,000-1,000,000 Daltons, preferably 10,000-500,000 Daltons, more preferably 20,000-250,000 Daltons. In preferred embodiments, the polyethylene glycol or a derivative thereof is within the range of 30,000-100,000 Daltons, for example, 40,000-80,000 Daltons. In one embodiment, the polyethylene glycol or a derivative thereof has a molecular weight about 50,000 Dalton. In preferred embodiments, the polyethylene glycol or a derivative thereof is a 50 kD 3-arm branched PEGs such as JPEG50K and NPEG50K shown in FIGS. 1A and 1B.

The specific characteristics of PEG moieties relevant to pharmaceutical applications include water solubility, high mobility in solution, lack of toxicity and low immunogenicity, ready clearance from the body, and altered distribution in the body.

PEGylation (also often styled pegylation) is the process of covalent and/or non-covalent attachment or amalgamation of polyethylene glycol (PEG) polymer chains to molecules and macrostructures, such as a small molecule drug, therapeutic protein or vesicle, which is then referred to as PEGylated (pegylated). PEGylation is routinely achieved by incubation of a reactive derivative of PEG with the target molecule. The covalent attachment of PEG to a drug or therapeutic protein can “mask” the agent from the host's immune system (reduced immunogenicity and antigenicity) and increase the hydrodynamic size (size in solution) of the agent which prolongs its circulatory time by reducing renal clearance. PEGylation can also provide water solubility to hydrophobic drugs and proteins.

PEGylation can improve the safety and efficiency of many therapeutics such as peptides, proteins, and antibody fragments. It produces alterations in the physiochemical properties including changes in conformation, electrostatic binding, hydrophobicity etc. These physical and chemical changes increase systemic retention of the therapeutic agent. Also, it can influence the binding affinity of the therapeutic moiety to the cell receptors and can alter the absorption and distribution patterns.

PEGylation increases molecular weight, defense of a metabolism site and inhibition of an immunogenicity site, increasing in vivo half-life and stability and reducing immunogenicity. Furthermore, kidney excretion of peptides and proteins bound with PEG is reduced due to the increase of molecular weights of peptides and proteins by PEG, so that PEGylation has advantages of increasing effects in both pharmacokinetically and pharmacodynamically.

In some embodiments, the GLP-1RA peptides are modified with polyethylene glycol, polyethyl imine, or derivatives thereof. In preferred embodiments, the GLP-1RA peptides are modified by PEGylation at the C-terminal cysteine residue. After the GLP-1RA peptide is PEGylated with polyethylene glycol or the derivative thereof is prepared, the molecular structure of the analogue may be confirmed by a mass spectroscope, a liquid chromatography, an X-ray diffraction analysis, a polarimetry, and comparison between calculated values and measured values of representative elements constituting the PEGylated GLP-1RA peptides.

In some embodiments, a PEG or a derivative thereof is conjugated to the GLP-1RA peptides via cysteine (C40) at the C-terminus of SEQ ID NOs: 1-7.

Exemplary PEGylated GLP-1RA peptides described in the Examples, have a 50 kD 3-arm branched PEGs (JPEG50K and NPEG50K) shown in FIGS. 1A and 1B. In some embodiments, PEGylated GLP-1RA peptides have the amino acids sequence and PEG conjugation as shown in one of SEQ ID NOs: 8-13 shown below:

(SEQ ID NO: 8)
HGEGTFTSDLSKQMEEEAVRLFIEWLKNGGPSSGAPPPSC(NPEG50K);
(SEQ ID NO: 9)
HGEGTFTSDLSKQMEEEAVRLFIEWLKNGGPSSGAPPPSC(JPEG50K);
(SEQ ID NO: 10)
HGEGTFTSDLSKQMEEEATRLFIEWLKNGGPSSGAPPPSC(JPEG50K);
(SEQ ID NO: 11)
HGEGTFTSDLSKQMEEEAARLFIEWLKNGGPSSGAPPPSC(JPEG50K);
(SEQ ID NO: 12)
HGEGTFTSDLSKQMEEEAVRLFGEWLKNGGPSSGAPPPSC(JPEG50K);
and
(SEQ ID NO: 13)
HGEGTFTSPLSKQMEEEAVRLFGEWLKNGGPSSGAPPPSC(JPEG50K).

### 3. Biological Activity of Modified GLP-1RA Peptides

### a. Biological Activity

Typically, the pegylated de-immunized GLP-1R agonists maintain bioactivity in vivo. As shown in Example 2, for example, the de-immunized GLP-1R agonists provided effective reductions in fasting blood glucose, HbA1c level, and improve glucose tolerance in diabetic mice. Further, the de-immunized GLP-1R agonists were effective in reducing body weight, fasting blood glucose, HbA1c level, and improving glucose tolerance in diabetic monkey.

In some embodiments, the de-immunized GLP-1R agonists show low immunogenicity T-cell proliferation assay compared to equivalent exenatide. In preferred embodiments, the GLP-1RA peptide analogs have EC₅₀ comparable to NLY01 (Exenatide-Cys-NPEG50K and Exenatide-Cys-JPEG50K) or exenatide, in cell-based GLP-1R cAMP assays.

In some embodiments, GLP-1RA peptide analogs have in vivo efficacy comparable to NLY01 (Exenatide-Cys-NPEG50K and Exenatide-Cys-JPEG50K) or other positive controls such as semaglutide in vivo studies, for example, using mouse models. In some embodiments, GLP-1RA peptide analogs have in vivo efficacy (e.g., reductions in blood glucose, HbA1c and ipGTT) comparable to NLY01 (Exenatide-Cys-NPEG50K) or Exenatide-Cys-JPEG50K or other positive controls such as semaglutide.

In preferred embodiments, the de-immunized GLP-1R agonists do not result in treatment-related effects on body weight, hematology, coagulation, or serum chemistry parameters. In some embodiments, the GLP-1RA peptide analogs (similarly modified as control, e.g., JPEG50K) have pharmacokinetic parameters (Cmax, AUC, T_{1/2}) comparable to NLY01 (Exenatide-Cys-NPEG50K) or Exenatide-Cys-JPEG50K.

### b. Low Immunogenicity

Typically, the de-immunized GLP-1R agonists have a lower immunogenicity in a host when compared to the immunogenicity of exenatide in the same host. Generally, the host is a mammal, preferably, human.

The lower immunogenicity may be a lower immunogenicity score for the de-immunized GLP-1R agonists when compared to that of exenatide. An exemplary in silico prediction of T cell epitopes such as MHC Class binding in GLP-1RAs immunogenicity hot spot is iTope-AI, which predicts peptide binding specificities across 46 major HLA-DR, DP and DQ Isotypes.

Typically, the de-immunized GLP-1R agonists produce minimal or lower levels of T cell activation markers in a host when compared to the level of T cell activation makers produced by exenatide in the same host, as detected by T cell assay or an equivalent assay. In preferred embodiments, comparison is carried out when the de-immunized GLP-1R agonists and exenatide are similarly modified, for example, both with conjugation via C-terminal cysteine to a 3-arm PEG of about 50 kDa.

In preferred embodiments, the de-immunized GLP-1R agonists typically have at least about 10%, 15%, 20%, 25%, 30%, 35%, 40%, 45%, or 50% lower total immunogenicity score when compared to exenatide or NLY01. For example, two peptide sequences of interest can be analyzed by predictive algorithm iTope-AI to identify peptides that bind to human MHC class II and/or share homology to known T cell epitopes. As described in Example 1, it was determined that SEQ ID NO:3, SEQ ID NO:4, and SEQ ID NO: 5 had a Total Score of 29, 19, and 27 respectively, with none of the peptide sequences matching a T cell epitope in T Cell Epitope Database (TCED™, Abzena database).

The currently available GLP-1R agonists such as exenatide (BYDUREON) are immunogenic with 43%-49% of patients developing antibodies and 6% with an attenuated glycemic response when administered once weekly. Such immune response to GLP-1RA is associated with reduced effectiveness in some patients and can also be associated with injection site reactions or, in some cases, a systemic allergic response. Thus, the lower immunogenicity of the de-immunized GLP-1R agonists provide a better and durable treatment for patients. In preferred embodiment, the de-immunized GLP-1R agonists maintain bioactivity while achieving an extended serum half-life of in humans, preferably more than 5 days, 6 days, 7 days, 8 days, 9 days, 10 days, 11 days, 12 days, 13 days, 14 days, or 15 days. In one embodiment, the extended serum half-life of in humans is more than 12 days.

### c. Pharmacokinetics

As discussed in more detail in Example 3, an earlier trial that illustrates the potential for a once-monthly GLP-1R agonist (Rosenstock J, Reusch J, Bush M, Yang F, Stewart M; Albiglutide Study Group. Diabetes Care. 2009 October; 32(10): 1880-6). Closer observation, however, revealed a substantial loss in fasting blood glucose control when albiglutide was administered monthly. PK data showing albiglutide concentrations fall essentially to zero between monthly doses. Monthly dosing was also associated with a significantly greater on dosing days than is observed at any time with weekly dosing.

Thus, in some embodiments, the therapeutically effective concentration of the de-immunized GLP-1RA agonist peptide is maintained in the serum of the subject at any time point during the treatment (peak, median, and trough) once a steady state is established after administration. In further embodiments, the concentration of the de-immunized GLP-1RA agonist peptide does not fall below pharmacologically active levels. In preferred embodiments, the continuous exposure to active levels of the GLP-1RA agonist peptide promotes GI tolerance when de-immunized GLP-1R agonist is administered monthly. In preferred embodiments, monthly dosing of the de-immunized GLP-1RA agonist peptide having any one of SEQ ID NOs: 7-13 provides minimal side effect, e.g., reducing incidence of nausea and vomiting observed in the earlier trial using albiglutide.

In one embodiment, the serum concentration of the de-immunized GLP-1RA agonist peptide is at or above 27 nM throughout the treatment once a steady state is established after administration.

In some embodiments, the GLP-1RA peptides and formulations thereof provide effective glycemic response after administration and the glycemic response is maintained for 1, 2, 3, 4, 5, 6, 7, 8, 9, 10, more than 10 years.

### B. Pharmaceutical Formulations

In some embodiments, the GLP-1RA peptides or analogs thereof are formulated with one or more pharmaceutical excipients, additives, or fillers. For example, in some embodiments, the GLP-1RA peptides or analogs thereof are formulated into pharmaceutical formulations for administration to a subject. Compositions including GLP-1RA peptides or analogs thereof may be formulated in a conventional manner using one or more physiologically acceptable carriers including excipients and auxiliaries which facilitate processing of the active compounds into preparations which can be used pharmaceutically.

Proper formulation is dependent upon the route of administration chosen. In preferred embodiments, the compositions are formulated for parenteral delivery. In preferred embodiments, the compositions are formulated for subcutaneous delivery. In some embodiments, the compositions are formulated for intravenous injection. Typically, the compositions will be formulated in sterile saline or buffered solution for injection into the tissues or cells to be treated. The compositions can be stored lyophilized in single use vials for rehydration immediately before use. Other means for rehydration and administration are known to those skilled in the art.

Pharmaceutical formulations contain the GLP-1RA peptides or analogs thereof in combination with one or more pharmaceutically acceptable excipients. Representative excipients include solvents, diluents, pH modifying agents, preservatives, antioxidants, suspending agents, wetting agents, viscosity modifiers, tonicity agents, stabilizing agents, and combinations thereof. Suitable pharmaceutically acceptable excipients are preferably selected from materials which are generally recognized as safe (GRAS), and may be administered to an individual without causing undesirable biological side effects or unwanted interactions.

Generally, pharmaceutically acceptable salts can be prepared by reaction of the free acid or base forms of an agent with a stoichiometric amount of the appropriate base or acid in water or in an organic solvent, or in a mixture of the two; generally, non-aqueous media like ether, ethyl acetate, ethanol, isopropanol, or acetonitrile are preferred. Pharmaceutically acceptable salts include salts of an agent derived from inorganic acids, organic acids, alkali metal salts, and alkaline earth metal salts as well as salts formed by reaction of the drug with a suitable organic ligand (e.g., quaternary ammonium salts). Lists of suitable salts are found, for example, in Remington's Pharmaceutical Sciences, 20th ed., Lippincott Williams & Wilkins, Baltimore, MD, 2000, p. 704. Examples of ophthalmic drugs sometimes administered in the form of a pharmaceutically acceptable salt include timolol maleate, brimonidine tartrate, and sodium diclofenac.

### 1. Dosage Units

The compositions are preferably formulated in dosage unit form for ease of administration and uniformity of dosage. The phrase “dosage unit form” refers to a physically discrete unit of conjugate appropriate for the patient to be treated. It will be understood, however, that the total single administration of the compositions will be decided by the attending physician within the scope of sound medical judgment. The therapeutically effective dose can be estimated initially either in cell culture assays or in animal models, usually mice, rats, rabbits, dogs, or pigs. The animal model is also used to achieve a desirable concentration range and route of administration. Such information should then be useful to determine useful doses and routes for administration in humans.

### 2. Formulations for Administration

In some embodiments, the compositions of GLP-1RA peptides or analogs thereof are formulated into a pharmaceutically acceptable formulation for administration via a specific route. In some embodiments, the compositions are administered locally, for example, by injection directly into a site to be treated. In some embodiments, the compositions are injected, topically applied, or otherwise administered directly into the vasculature onto vascular tissue at or adjacent to a site of injury, surgery, or implantation. For example, in some embodiments, the compositions are topically applied to vascular tissue that is exposed, during a surgical procedure. Typically, local administration causes an increased localized concentration of the compositions, which is greater than that which can be achieved by systemic administration.

Pharmaceutical compositions formulated for administration by parenteral (intramuscular, intraperitoneal, intravenous (IV) or subcutaneous injection) and enteral routes of administration are described.

### a. Enteral Administration

In some embodiments, the GLP-1RA peptides or analogs thereof are administered orally. For oral administration, suitable formulations include tablets, pellets, hard/soft capsules, liquids, suspensions, emulsifiers, syrups, granules, elixirs, troches, etc., and these formulations can include diluents (for example, lactose, dextrose, sucrose, mannitol, sorbitol, cellulose and/or glycine), slip modifiers (for example, silica, talc, stearate and its magnesium or calcium salt and/or polyethylene glycol) in addition to the active ingredient. Tablets may also include binders such as magnesium aluminum silicate, starch paste, gelatin, methyl cellulose, sodium carboxymethyl cellulose and/or polyvinyl pyrrolidine, and may include disintegrating agents such as starch, agar, alginic acid or sodium salt thereof or boiling mixture and/or absorbents, coloring agents, flavoring agents and sweetening agents if needed.

In preferred embodiments, one or more absorption or permeation enhancers are used for oral formulation. Exemplary permeation enhancers include bile acid, cholic acid, deoxycholic acid, glycocholic acid, glycochonodeoxycholic acid, taurochenodeoxycholic acid, taurocholic acid, chenodeoxycholic acid, ursodeoxycholic acid, lithocholic acid, Labrasol (Caprylocaproyl Polyoxyl-8 glycerides), SNAC (sodium N-(8-[2-hydroxybenzoyl]amino) caprylate and their salt forms.

### b. Parenteral Administration

In some embodiments, the GLP-1RA peptides or analogs thereof are formulated into a pharmaceutically acceptable formulation for parenteral administration. The phrases “parenteral administration” and “administered parenterally” are art-recognized terms, and include modes of administration other than enteral and topical administration, such as injections, and include without limitation intravenous (i.v.), intramuscular (i.m.), intraperitoneal (i.p.), subcutaneous (s.c.) injection and infusion. The long-acting GLP-1r agonists can be administered parenterally, for example, by intravenous, intraperitoneal, or subcutaneous routes.

For liquid formulations, pharmaceutically acceptable carriers may be, for example, aqueous or non-aqueous solutions, suspensions, emulsions, or oils. Parenteral vehicles (for subcutaneous, intravenous, intraarterial, or intramuscular injection) include, for example, sodium chloride solution, Ringer's dextrose, dextrose and sodium chloride, lactated Ringer's and fixed oils. Examples of non-aqueous solvents are propylene glycol, polyethylene glycol, and injectable organic esters such as ethyl oleate. Aqueous carriers include, for example, water, alcoholic/aqueous solutions, cyclodextrins, emulsions or suspensions, including saline and buffered media. The long-acting GLP-IRAs can also be administered in an emulsion, for example, water in oil. Examples of oils are those of petroleum, animal, vegetable, or synthetic origin, for example, peanut oil, soybean oil, mineral oil, olive oil, sunflower oil, fish-liver oil, sesame oil, cottonseed oil, corn oil, olive, petrolatum, and mineral. Suitable fatty acids for use in parenteral formulations include, for example, oleic acid, stearic acid, and isostearic acid. Ethyl oleate and isopropyl myristate are examples of suitable fatty acid esters.

Formulations suitable for parenteral administration can include antioxidants, buffers, bacteriostats, and solutes that render the formulation isotonic with the blood of the intended recipient, and aqueous and non-aqueous sterile suspensions that can include suspending agents, solubilizers, thickening agents, stabilizers, and preservatives. Intravenous vehicles can include fluid and nutrient replenishers, electrolyte replenishers such as those based on Ringer's dextrose. In general, water, saline, aqueous dextrose and related sugar solutions, and glycols such as propylene glycols or polyethylene glycols are preferred liquid carriers, particularly for injectable solutions.

Injectable pharmaceutical carriers for injectable compositions are well-known to those of ordinary skill in the art (see, e.g., Pharmaceutics and Pharmacy Practice, J.B. Lippincott Company, Philadelphia, PA, Banker and Chalmers, eds., pages 238-250 (1982), and ASHP Handbook on Injectable Drugs, Trissel, 15th ed., pages 622-630 (2009)).

### III. Methods of Selecting GLP-1RA Peptide Analogs

A. Methods of Screening and Identifying GLP-1RA Peptide Analogs with Reduced Immunogenicity

To identify one or more new GLP-1RA peptide analogs having reduced immunogenicity but maintained in vivo efficacy compared to exenatide (e.g., BYDUREON®), one or more following assays can be carried out.

In some embodiments, the methods involve the step of in silico prediction of T cell epitopes such as MHC Class binding in GLP-1RAs immunogenicity hot spot. Exemplary algorithms include iTope-AI, which predicts peptide binding specificities across 46 major HLA-DR, DP and DQ Isotypes. In other embodiments, the methods further involve the step of in silico validation of the immunogenicity of one or more variants having amino acid substitutions that were predicted to have reduced MHC Class II binding.

In silico prediction of T-Cell epitopes provides the initial step to assess immunogenicity potential, it does not account for all factors that influence clinical immunogenicity such as antigen processing, T cell receptor recognition of the MHC class II/peptide complex, T cell tolerance to any non-germline peptide, protein structure, formulation, pharmacology, and patient history, etc. Therefore, in some embodiments, immunogenicity potential is assayed using in vitro assay. Exemplary assays include T-cell proliferation assay as shown in Example 1. In one embodiment, a population of donors are selected to best represent the number and frequency of HLA-DR and HLA-DQ allotypes expressed in European/North American and the world population. In some embodiments, HLA-DP is not considered in the selection, due to its more limited polymorphism and likely low levels of expression. In some embodiments, bulk cultures are established using CD8+ T cell depleted PBMCs, and CD4+ T cell proliferation is measured at various time points after the addition of the sample by incorporation of [3H]-Thymidine. In preferred embodiments, GLP-1RA peptide analogs with low immunogenicity are selected based on the response rate in the T-cell proliferation assay.

In some embodiments, potency of the new GLP-1RA peptide analog is evaluated in cell-based GLP-1R cAMP assay. GLP-1R agonists can activate the adenylyl cyclase pathway resulting in increased intracellular levels of cAMP. In one embodiment, stable GLP-1R expressing HEK293 cells co-transfected with a luciferase gene controlled by a cAMP responsive element CRE-luc are used in this potency assay. Expression level of the luciferase gene is directly proportional to the intracellular cAMP produced upon stimulation of the GLP1-R by GLP-1RA peptide analog and/or modified variants thereof. In some embodiments, luminescence is measured using One-Glo™ luciferase assay system. The biological activity of GLP-1RA peptide analog and/or modified variants thereof is expressed as the effective concentration inducing 50% stimulation of the maximum response (EC₅₀). In preferred embodiments, GLP-1RA peptide analogs with EC₅₀ comparable to NLY01 (Exenatide-Cys-NPEG50K and Exenatide-Cys-JPEG50K) are selected.

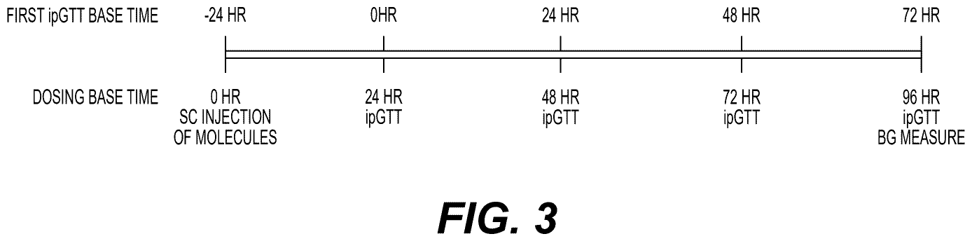

In some embodiments, glycemic control efficacy is evaluated by intraperitoneal glucose tolerance test (ipGTT) in mice, for example C57BL/6 male mice. In one embodiment, ipGTT tests are conducted at 24, 48, and 72 hours after a single subcutaneous dose as shown in FIG. 3. In preferred embodiments, GLP-1RA peptide analogs with in vivo efficacy comparable to NLY01 (Exenatide-Cys-NPEG50K and Exenatide-Cys-JPEG50K) or other positive controls such as semaglutide are selected.

In some embodiments, glycemic control efficacy is evaluated using db/db mice model as demonstrated in Example 2. Leptin receptor-deficient db/db mice are commonly used mice models mimicking the conditions of obesity and type 2 diabetes development. Mice that are homozygous for the diabetes spontaneous mutation (Lepr^{db}) demonstrate morbid obesity, chronic hyperglycemia, pancreatic beta cell atrophy and come to be hypoinsulinemic. In one embodiment, after a single dose via subcutaneous injection, fed blood glucose is measured at 0 through 96 hours. In another embodiment, a twice weekly dose in administered and fasting blood glucose is measure weekly. In a further embodiment, HbA1c and ipGTT are also evaluated. In some embodiments, GLP-1RA peptide analogs with in vivo efficacy (e.g., reductions in blood glucose) comparable to NLY01 (Exenatide-Cys-NPEG50K) or Exenatide-Cys-JPEG50K or other positive controls such as semaglutide are selected. In preferred embodiments, in vivo assays to evaluate glycemic control efficacy are used to establish effective dosages. Thus, in some embodiments, the dosage of GLP-1RA peptide analogs with more effective reduction in fasting blood glucose, HbA1c and ipGTT is selected.

In some embodiments, pharmacokinetics (Cmax, AUC, T_{1/2}) are evaluated in non-human primates. In one embodiment, a single subcutaneous dose is given. In another embodiment, blood sample is collected pre-dose, then at multiple time-points after the administration (e.g., 1, 4, 8, 24, 48, 72, 120, 168 hours after dosing, then again on Days 15 and Day 22) for pharmacokinetic analysis. In preferred embodiments, the dosage does not result in treatment-related effects on body weight, hematology, coagulation, or serum chemistry parameters. In some embodiments, GLP-1RA peptide analogs with pharmacokinetic parameters (Cmax, AUC, T_{1/2}) comparable to NLY01 (Exenatide-Cys-NPEG50K) or Exenatide-Cys-JPEG50K are selected and identified as a new GLP-1RA peptide analogs having reduced immunogenicity but maintained in vivo efficacy.

In further embodiments, therapeutic efficacy is evaluated in Obese/T2D non-human primates for clinical development. In one embodiment, the effect of the GLP-1RA peptide analog (e.g., NLY12) is evaluated in obese/diabetic male cynomolgus monkeys. These monkeys develop type 2 diabetes and obesity naturally through age and diet, providing a non-human primate model for disease. In one embodiment, this study evaluates safety and tolerability of the GLP-1RA peptide analog following weekly or biweekly subcutaneous injection, and evaluates the effect on fasting glucose, glucose tolerance, body weight, and HbA1c. In one embodiment, other parameters measured include food intake and clinical chemistry. In some embodiments, the GLP-1RA peptide analog with effective reduction in fasting glucose, food intake, body weight, and HbA1c, and/or improve glucose tolerance are selected and identified as a new GLP-1RA peptide analogs having reduced immunogenicity but maintained in vivo efficacy. In preferred embodiments, the GLP-1RA peptide analog maintains steady concentration in serum (peak, median, and trough) all overlap with known therapeutic concentration of existing exenatide therapeutics such as semaglutide.

In some embodiments, the methods further include the step of administering an effective amount of the new GLP-1RA peptide analogs having reduced immunogenicity but maintained in vivo efficacy, to a subject in need thereof. In preferred embodiments, the GLP-1RA peptide analog is administered once a month while maintaining steady concentration in serum (peak, median, and trough) all overlap with known therapeutic concentration of existing exenatide therapeutics such as semaglutide.

### B. Methods of Making GLP-1RA Peptide Analogs

Methods of making recombinant peptides are known in the art. Modifications to GLP-1RAs can be prepared and modified via a variety of chemical reaction steps. Typically, methods for modifying the GLP-1RAs include pegylation.

### IV. Methods of Use

Pharmaceutical formulations containing one or more of the GLP-1RA peptides or analogs thereof can be administered to a subject in need thereof to treat or prevent one or more diseases.

### A. Methods of Treatment

Methods of using GLP-1RA peptides for treating or preventing one or more metabolic diseases are described. Methods are effective in treating or preventing one or more metabolic diseases such as dyslipidemia, fatty liver disease, metabolic syndrome, nonalcoholic fatty liver disease (NAFLD), obesity and type 2 diabetes mellitus (T2DM), preferably with minimal side effects.

In preferred embodiments, the GLP-1RA peptides or analogs thereof are administered in an amount and with a dosing regimen effective to prevent, inhibit, or reduce one or more symptoms associated with one or more metabolic diseases such as dyslipidemia, fatty liver disease, metabolic syndrome, NAFLD, obesity and T2DM in the subject. In preferred embodiments, the disease is fatty liver disease, obesity, NAFLD or T2DM.

The GLP-1RA peptides or analogs thereof are administered to a subject in one or multiple doses, at one or multiple time points following an initial dose. The amount of compositions administered to the subject is selected to deliver an effective amount to reduce, prevent, or otherwise alleviate one or more clinical or molecular symptoms of the disease or disorder to be treated compared to a control, for example, a subject treated with an agonist primarily targeting a single receptor of GLP-1. The compositions and methods are also suitable for prophylactic use.

Methods are also suitable for treating or preventing one or more neurodegenerative diseases such as Alzheimer's Disease (AD) or Parkinson's disease. In some embodiments, methods include a step of administering to an individual in need thereof an effective amount of a GLP-1RA peptide or analog thereof.

### B. Conditions to be Treated

The compositions and formulations of these GLP-1RA peptides are effective to alleviate or prevent one or more symptoms of metabolic diseases or neurological diseases with minimal off-target toxicity or side effects.

In some embodiments, the subject to be treated is a human. In some embodiments, the subject to be treated is a child, or an infant. All the methods can include the step of identifying and selecting a subject in need of treatment, or a subject who would benefit from administration with the described compositions.

### 1. Obesity and Type 2 Diabetes Mellitus

Obesity is a medical condition in which excess body fat has accumulated to the extent that it may have an adverse effect on health, leading to reduced life expectancy and/or increased health problems. The most accurate method for evaluation of obesity is to measure body fat mass. However, accurate measurement of fat mass is costly, and thus it is evaluated using indirect methods. The most commonly used indirect methods are to measure body mass index (BMI) and waist circumference. BMI, a measurement which compares weight and height, defines people as overweight (pre-obese or overweight) if their BMI is between 25 and 30 kg/m², and obese when it is greater than 30 kg/m².

Obesity increases the risk of many physical and mental disorders. Excessive body weight is associated with various diseases, particularly cardiovascular diseases, diabetes mellitus type 2, obstructive sleep apnea, certain types of cancer, and osteoarthritis. These diseases are either directly caused by obesity or indirectly related through mechanisms sharing a common cause such as a poor diet and/or a sedentary lifestyle. One of the strongest links is with type 2 diabetes. Excess body fat underlies 64% of cases of diabetes in men and 77% of cases in women. Increases in body fat alter the body's response to insulin, potentially leading to insulin resistance.

Methods to treat and/or prevent one or more symptoms of obesity and/or T2DM include administering to a subject in a need thereof an effective amount of a composition to treat and/or alleviate one or more symptoms associated with obesity and/or T2DM. In some embodiments, the compositions or pharmaceutical formulations thereof are administered in an amount effective to induce weight loss, reduce body fat, reduce food intake, improve glucose homeostasis, prevent weight gain, and/or prevent an increase in body mass index in a normal, or obese patient, or combinations thereof.

In some embodiments, the pharmaceutical formulations are administered to a patient suffering from obesity, an obesity-related disease or disorder, diabetes, insulin-resistance syndrome, nonalcoholic steatohepatitis, a cardiovascular disease, or a metabolic syndrome.

In some embodiments, the pharmaceutical formulations are administered to normalize blood sugar; the formulations are preferably administered in an amount effective to lower blood glucose levels to less than about 180 mg/dL. The formulations can be co-administered with other anti-diabetic therapies, if necessary, to improve glucose homeostasis.

Pharmaceutical formulations may also be administered to patients suffering from a disease or disorder that causes obesity or predisposes a patient to become obese.

### 2. Non-Alcoholic Fatty Liver Disease (NAFLD)

In preferred embodiments, the methods and compositions are used to treat or prevent non-alcoholic steatohepatitis, liver fibrosis associated with non-alcoholic steatohepatitis, primary biliary cholangitis.

In some embodiments, the compositions are used to treat nonalcoholic fatty liver disease (NAFLD). NAFLD represents a clinico-pathological spectrum of disease that primarily manifests as excessive accumulation of fat in the hepatocyte (steatosis). NAFLD encompasses the entire spectrum of diseases ranging from simple steatosis to non-alcoholic steatohepatitis (NASH), which can lead to life-threatening hepatic cirrhosis and hepatocellular carcinoma in its most severe form. It is considered to be the hepatic manifestation of the metabolic syndrome, whose other pathologies include obesity, insulin resistance, hypertension and hyperlipidemia. Histologically, NASH is characterized by hepatic steatosis and signs of intralobular inflammation with ballooning degeneration of the hepatocytes. The estimated prevalence of NASH is much lower than NAFLD and ranges from 3 to 5%. Twenty percent of NASH patients are reported to develop cirrhosis, and 30-40% of patients with NASH cirrhosis experience a liver related death.

NAFLD is broadly categorized into 2 phenotypes, namely: non-alcoholic fatty liver (NAFL) which is marked by isolated steatosis, while the more aggressive subtype, non-alcoholic steatohepatitis (NASH), is characterized by cell injury, inflammatory cell infiltration and hepatocyte ballooning that may further progress to fibrosis, cirrhosis, and hepatocellular carcinoma (HCC). In some embodiments, the compositions are used in an amount effective for treating or ameliorating one or more symptoms of non-alcoholic steatohepatitis (NASH).

NAFLD shows a close association with the metabolic syndromes including obesity, type II diabetes, dyslipidemia, and the like based on insulin resistance. In fact, it is known that many pre-diabetic and type II diabetic patients have shown to present with non-alcoholic fatty liver/non-alcoholic steatohepatitis, and the rate of progression to liver cirrhosis and liver cancer (i.e., hepatocellular carcinoma) is high in these patients. Meanwhile, the prevalence of diabetes in non-alcoholic fatty liver disease patients is high, and it is evident in non-alcoholic steatohepatitis patients.

The non-alcoholic fatty liver disease may include one or more diseases selected from the group consisting of non-alcoholic fatty liver, non-alcoholic steatohepatitis, liver cirrhosis, and liver cancer.

In some embodiments, the compositions are administered in an amount effective to prevent the transformation of NAFLD into NASH and to improve the pathophysiology of the disease.

Methods to treat and/or prevent one or more symptoms of NAFLD or NASH typically include administering to a subject in a need thereof an effective amount of a composition to treat and/or alleviate one or more symptoms associated with NAFLD or NASH.

In some embodiments, the compositions are administered in an amount effective to inhibit or reduce serum levels of alanine aminotransferase (ALT), aspartate aminotransferase (AST), triglyceride (TG) and total cholesterol (TC), fat accumulation or steatosis, inflammation, ballooning, fibrosis, long-term morbidity, and mortality.

### 3. Neurological and Neurodegenerative Diseases

The compositions and formulations thereof can be used to treat one or more neurological and neurodegenerative diseases. The compositions and methods are particularly suited for treating one or more neurological, or neurodegenerative diseases associated with activation of microglia and/or astrocytes. In some embodiments, the disease or disorder is selected from, but not limited to, neurological disorders (e.g., Alzheimer's disease (AD), Parkinson's disease (PD)). In one embodiment, the compositions are used to treat Alzheimer's Disease (AD) or Parkinson's disease.

Neurodegenerative diseases are chronic progressive disorders of the nervous system that affect neurological and behavioral function and involve biochemical changes leading to distinct histopathologic and clinical syndromes (Hardy H, et al., Science. 1998; 282:1075-9). Abnormal proteins resistant to cellular degradation mechanisms accumulate within the cells. The pattern of neuronal loss is selective in the sense that one group gets affected, whereas others remain intact. Often, there is no clear inciting event for the disease. The diseases classically described as neurodegenerative are Alzheimer's disease, Huntington's disease, and Parkinson's disease.

Neuroinflammation, mediated by activated microglia and astrocytes, is a major hallmark of various neurological disorders making it a potential therapeutic target. Multiple scientific reports suggest that mitigating neuroinflammation in early phase by targeting these cells can delay the onset of disease and can in turn provide a longer therapeutic window for the treatment (Dommergues, M A et al., Neuroscience 2003, 121, 619; Perry, V H et al., Nat Rev Neurol 2010, 6, 193; Kannan, S et al., Sci. Transl. Med. 2012, 4, 130ra46; and Block, M L et al., Nat Rev Neurosci 2007, 8, 57). The delivery of therapeutics across blood brain barrier is a challenging task. The neuroinflammation causes disruption of blood brain barrier (BBB). The impaired BBB in neuroinflammatory disorders can be utilized to transport drug loaded nanoparticles across the brain (Stolp, H B et al., Cardiovascular Psychiatry and Neurology 2011, 2011, 10; and Ahishali, B et al., International Journal of Neuroscience 2005, 115, 151).

The compositions and methods can also be used to for the treatment of a neurological or neurodegenerative disease or disorder or central nervous system disorder. In preferred embodiments, the compositions and methods are effective in treating, and/or alleviating neuroinflammation associated with a neurological or neurodegenerative disease or disorder or central nervous system disorder. The methods typically include administering to the subject an effective amount of the composition to increase cognition or reduce a decline in cognition, increase a cognitive function or reduce a decline in a cognitive function, increase memory or reduce a decline in memory, increase the ability or capacity to learn or reduce a decline in the ability or capacity to learn, or a combination thereof.

Neurodegeneration refers to the progressive loss of structure or function of neurons, including death of neurons. For example, the compositions and methods can be used to treat subjects with a disease or disorder, such as Parkinson's Disease (PD) and PD-related disorders, Huntington's Disease (HD), Amyotrophic Lateral Sclerosis (ALS), Alzheimer's Disease (AD) and other dementias, Prion Diseases such as Creutzfeldt-Jakob Disease, Corticobasal Degeneration, Frontotemporal Dementia, HIV-Related Cognitive Impairment, Mild Cognitive Impairment, Motor Neuron Diseases (MND), Spinocerebellar Ataxia (SCA), Spinal Muscular Atrophy (SMA), Friedreich's Ataxia, Lewy Body Disease, Alpers' Disease, Batten Disease, Cerebro-Oculo-Facio-Skeletal Syndrome, Corticobasal Degeneration, Gerstmann-Straussler-Scheinker Disease, Kuru, Leigh's Disease, Monomelic Amyotrophy, Multiple System Atrophy, Multiple System Atrophy With Orthostatic Hypotension (Shy-Drager Syndrome), Multiple Sclerosis (MS), Neurodegeneration with Brain Iron Accumulation, Opsoclonus Myoclonus, Posterior Cortical Atrophy, Primary Progressive Aphasia, Progressive Supranuclear Palsy, Vascular Dementia, Progressive Multifocal Leukoencephalopathy, Dementia with Lewy Bodies (DLB), Lacunar syndromes, Hydrocephalus, Wernicke-Korsakoff's syndrome, post-encephalitic dementia, cancer and chemotherapy-associated cognitive impairment and dementia, and depression-induced dementia and pseudodementia. In preferred embodiments, the disease or disorder is Alzheimer's Disease (AD) or Parkinson's disease.

Criteria for assessing improvement in a particular neurological factor include methods of evaluating cognitive skills, motor skills, memory capacity or the like, as well as methods for assessing physical changes in selected areas of the central nervous system, such as magnetic resonance imaging (MRI) and computed tomography scans (CT) or other imaging methods. Such methods of evaluation are well known in the fields of medicine, neurology, psychology and the like, and can be appropriately selected to diagnosis the status of a particular neurological impairment. To assess a change in Alzheimer's disease, or related neurological changes, the selected assessment or evaluation test, or tests, are given prior to the start of administration of the compositions. Following this initial assessment, treatment methods for the administration of the compositions are initiated and continued for various time intervals. At a selected time interval subsequent to the initial assessment of the neurological defect impairment, the same assessment or evaluation test(s) is again used to reassess changes or improvements in selected neurological criteria.

The individual is preferably an adult human, and more preferably, a human is over the age of 30, who has lost some amount of neurological function as a result of Alzheimer's disease or dementia. Generally, neural loss implies any neural loss at the cellular level, including loss in neurites, neural organization, or neural networks.

In other embodiments, the methods including selecting a subject who is likely to benefit from treatment with the compositions.

The compositions and formulations are suitable for reducing or preventing one or more pathological processes associated with the development and progression of PD. Thus, methods for treatment, reduction, and prevention of the pathological processes associated with PD include administering the compositions in an amount and dosing regimen effective to reduce microglial activation, abnormal accumulation of alpha-synuclein protein, neurofibrillary tangles in brains, and/or improved shaking, rigidity, slowness of movement and difficulty with walking, in an individual suffering from PD are provided. Methods for reducing, preventing, or reversing the motor dysfunction in an individual suffering from PD are provided. The methods include administering an effective amount of a composition including one or more long-acting GLP-1r agonists to a subject in need thereof. In preferred embodiments, the methods include administering an effective amount of a composition including one or more GLP-1RA peptides having amino acid sequence of any one of SEQ ID NOs: 1-13, or pharmaceutically acceptable salt thereof to the subject.

### C. Dosage and Effective Amounts

Dosage and dosing regimens are dependent on the severity of the disorder and/or methods of administration, and can be determined by those skilled in the art. A therapeutically effective amount of GLP-1RA peptides, or pharmaceutical formulation thereof used in the treatment of fatty liver disease, metabolic syndrome, nonalcoholic fatty liver disease (NAFLD), obesity and/or type 2 diabetes mellitus (T2DM) is typically sufficient to reduce or alleviate one or more symptoms associated with the disease or disorder.

Preferably, the compositions do not target or otherwise modulate the activity or quantity of healthy cells not within or associated with the diseased or target tissues, or do so at a reduced level compared to target cells. In this way, by-products and other side effects associated with the compositions are reduced.

The actual effective amounts can vary according to factors including the specific agent administered, the particular composition formulated, the mode of administration, and the age, weight, condition of the subject being treated, as well as the route of administration and the disease or disorder. In some embodiments, the dose of the GLP-1RA analog, or pharmaceutical formulation thereof can be from about 0.01 to about 100 mg/kg body weight, from about 0.01 mg/kg to about 10 mg/kg, and from about 0.05 mg to about 5 mg/kg body weight. In other embodiments, the dosage is an absolute amount of a GLP-1RA analog, or pharmaceutical formulation thereof, for a single administration to a subject, such as from about 0.1 mg up to about 100 mg. For example, in some embodiments, the dosage of a GLP-1RA analog, or pharmaceutical formulation thereof is 0.1 mg, 0.5 mg, 1 mg, 2 mg, 3 mg, 4 mg, 5 mg, 6 mg, 7 mg, 8 mg, 9 mg or 10 mg, or more than 10 mg, for example 20 mg, 30 mg, 40 mg, 50 mg, or 100 mg.

In an exemplary embodiment, the dosage of GLP-1RA analog is between about 5 mg and about 40 mg, administered once a month. Generally, for intravenous injection or infusion, the dosage may be lower than for oral administration.

In general, the timing and frequency of administration will be adjusted to balance the efficacy of a given treatment schedule with the side-effects of the given delivery system. Exemplary dosing frequencies include continuous infusion, single and multiple administrations such as weekly, monthly, or yearly dosing.

The GLP-1RA analog, or pharmaceutical formulation thereof can be administered daily, biweekly, weekly, every two weeks, monthly, or less frequently in an amount to provide a therapeutically effective increase in the blood level of the therapeutic agent. Where the administration is by other than an oral route, the compositions may be delivered over a period of more than one hour, e.g., 3-10 hours, to produce a therapeutically effective dose within a 24-hour period. Alternatively, the compositions can be formulated for controlled release, wherein the composition is administered as a single dose that is repeated on a regimen of once a week, or less frequently.

Dosage can vary, and can be administered in one or more doses once a week, once every two weeks, once monthly, once every two months, or less frequently. Guidance can be found in the literature for appropriate dosages for given classes of pharmaceutical products. Optimal dosing schedules can be calculated from measurements of drug accumulation in the body of the subject or patient. Persons of ordinary skill can easily determine optimum dosages, dosing methodologies and repetition rates. Optimum dosages can vary depending on the relative potency of individual pharmaceutical compositions, and can generally be estimated based on EC₅₀S found to be effective in in vitro and in vivo animal models.

In some embodiments, the compositions are administered to a subject for between 1 to 20 years, e.g., 1 year, 2 years, 3 years, 4 years, 5 years, 6 years, 7 years, 8 years, 9 years, 10 years, 11 years, 12 years, 13 years, 14 years, 15 years, 16 years, 17 years, 18 years, 19 years, or 20 years.

Optionally, the compositions are administered for 10 years. In one embodiment, the effects of treatment last for at least 1 year.

In preferred embodiments, the GLP-1RA analog is administered orally in an amount between about 1 mg and about 100 mg, inclusive, preferably between about 5 mg and about 50 mg, inclusive. In some embodiments, the GLP-1RA peptide or analog thereof is administered orally once a week, once every two weeks, once monthly, or less frequently. In preferred embodiments, the GLP-1RA peptide or analog thereof is administered orally once monthly, once every two months, or less frequently. In one embodiment, the GLP-1RA peptides and formulations thereof are suitable for once monthly administration.

In other preferred embodiments, the GLP-1RA analog is administered parentally such as subcutaneously at a concentration between about 0.1 mg/mL and about 10 mg/mL, inclusive, preferably between about 1 mg/mL and about 5 mg/mL, inclusive. In some embodiments, the GLP-1RA analog is administered parentally daily, biweekly, weekly, every two weeks, monthly, or less frequently. In preferred embodiments, the GLP-1RA analog having the amino acid sequence of any one of SEQ ID NOs: 1-13 is administered parentally once a month, once every two months, or less frequently. In one embodiment, the GLP-1RA analog having the amino acid sequence of any one of SEQ ID NOs: 1-13 is administered parentally once a month while maintaining therapeutic effective concentration in the serum at any time point during the treatment (peak, median, and trough) once a steady state is established.

In some embodiments, the regimen includes one or more cycles of a round of therapy followed by a drug holiday (e.g., no drug). The drug holiday can be 1, 2, 3, 4, 5, 6, or 7 days, or 1, 2, 3, 4 weeks, or 1, 2, 3, 4, 5, or 6 months.

In some embodiments, the amount of a GLP-1RA analog administered to a subject changes over time following an initial dose.

### D. Combination Therapies and Procedures

The compositions can be administered alone or in combination with one or more conventional therapies. Examples of preferred additional therapeutic agents include other conventional therapies known in the art for treating the desired disease, disorder, or condition. The additional therapeutic, prophylactic or diagnostic agent(s) can have the same or different mechanisms of action. In some embodiments, the combination results in an additive effect on the treatment of the disease or condition. In some embodiments, the combinations result in a more than additive effect on the treatment of the disease or disorder.

### E. Controls

The therapeutic result of the GLP-1RA analog, or pharmaceutical formulation thereof can be compared to a control or reference, for example, exenatide with a similar PEG modification (e.g., NLY01). The terms “control” or “reference” refer to a standard of comparison. The term “changed as compared to a control” sample or subject is understood as having a level that is statistically different than a sample from a normal, untreated, or control sample. Control samples include, for example, cells in culture, one or more laboratory test animals, or one or more human subjects. Methods to select and test control samples are within the ability of those in the art. An analyte can be a naturally occurring substance that is characteristically expressed or produced by the cell or organism (e.g., an antibody, a protein) or a substance produced by a reporter construct (e.g., β-galactosidase or luciferase). Depending on the method used for detection, the amount and measurement of the change can vary. Determination of statistical significance is within the ability of those skilled in the art, e.g., the number of standard deviations from the mean that constitute a positive result. Suitable controls are known in the art and include, for example, an untreated subject or untreated cells or the same individual prior to treatment.

### V. Kits

The compositions can be packaged in kit. The kit can include a single dose or a plurality of doses of a composition including one or more of the GLP-1RA peptides or analogs thereof, or pharmaceutical formulation thereof, and instructions for administering the compositions. In preferred embodiments, the GLP-1RA peptides or analogs thereof have the amino acid sequence of any one of SEQ ID NOs: 1-13. Specifically, the instructions direct that an effective amount of the composition be administered to an individual with a particular symptoms, disease, defect, or impairment as indicated. The composition can be formulated as described above with reference to a particular treatment method and can be packaged in any convenient manner.

The present invention will be further understood by reference to the following non-limiting examples.

### EXAMPLES

### Example 1: Development and Production of Exenatide variants

### Methods

### In Silico Prediction of MHC Class Binding in Exenatide-Cys Sequence

In order to reduce the immunogenicity potential of NLY01, potential T-cell epitopes in the exenatide-Cys (i.e., NLY01 peptide intermediate) amino acid sequence were identified. Valine at the 19th position (V19) was identified as the major contributor of the epitope score, with alanine, isoleucine, leucine, methionine, and threonine as feasible substitutions, illustrated in Table 1.

TABLE 1
Wide-type and variant of exenatide and their epitope scores.
wild type; total epitope score = 36

| | | | | | | | | | | | | | | | | | | | | | | |
|---|---|---|---|---|---|---|---|---|---|---|---|---|---|---|---|---|---|---|---|---|---|---|
| wild | H | G | E | G | T | F | T | S | D | L | S | K | Q | M | E | E | E | A | V | R | L | F |
| type | | | | | | | | | | | | | | | | | | | | | | |
| epitope | 0 | 0 | 0 | 0 | 0 | 5 | 0 | 0 | 0 | 0 | 0 | 0 | 0 | 0 | 0 | 0 | 0 | 0 | 19 | 0 | 1 | 4 |
| scores | | | | | | | | | | | | | | | | | | | | | | |
| allowed | H | G | D | G | S | F | S | A | D | F | A | E | E | I | D | D | D | A | A | K | F | F |
| residues | | | E | | T | Y | T | D | E | I | D | K | K | L | E | E | E | G | I | Q | I | Y |
| | | | K | | | | | E | | L | E | Q | Q | M | K | K | K | S | L | R | L | |
| | | | Q | | | | | G | | M | G | R | R | V | Q | Q | Q | T | M | | M | |
| | | | | | | | | K | | V | K | | | | | | | V | T | | V | |
| | | | | | | | | N | | | N | | | | | | | | V | | | |
| | | | | | | | | Q | | | Q | | | | | | | | | | | |
| | | | | | | | | S | | | S | | | | | | | | | | | |
| | | | | | | | | T | | | T | | | | | | | | | | | |

1 substitutions; total epitope score = 17

| | | | | | | | | | | | | | | | | | | | | | | |
|---|---|---|---|---|---|---|---|---|---|---|---|---|---|---|---|---|---|---|---|---|---|---|
| variant | H | G | E | G | T | F | T | S | D | L | S | K | Q | M | E | E | E | A | T | R | L | F |
| epitope | 0 | 0 | 0 | 0 | 0 | 5 | 0 | 0 | 0 | 0 | 0 | 0 | 0 | 0 | 0 | 0 | 0 | 0 | 0 | 0 | 1 | 4 |
| scores | | | | | | | | | | | | | | | | | | | | | | |
| variant | H | G | E | G | T | F | T | S | D | L | S | K | Q | M | E | E | E | A | A | R | L | F |
| epitope | 0 | 0 | 0 | 0 | 0 | 5 | 0 | 0 | 0 | 0 | 0 | 0 | 0 | 0 | 0 | 0 | 0 | 0 | 0 | 0 | 1 | 4 |
| scores | | | | | | | | | | | | | | | | | | | | | | |

| | wild type; total epitope score = 36 | | | | | | | | | | | | | | | | | |
|---|---|---|---|---|---|---|---|---|---|---|---|---|---|---|---|---|---|---|
| wild | I | E | W | L | K | N | G | G | P | S | S | G | A | P | P | P | S | C |
| type | | | | | | | | | | | | | | | | | | |
| epitope | 0 | 0 | 4 | 3 | 0 | 0 | 0 | 0 | 0 | 0 | 0 | 0 | 0 | 0 | 0 | 0 | 0 | 0 |
| scores | | | | | | | | | | | | | | | | | | |
| allowed | F | D | W | F | E | N | G | G | P | A | A | G | A | P | P | P | A | C |
| residues | I | E | | I | K | | | | | D | D | | G | | | | D | |
| | L | K | | L | Q | | | | | E | E | | S | | | | E | |
| | M | Q | | M | R | | | | | G | G | | T | | | | G | |
| | V | | | V | | | | | | K | K | | V | | | | K | |
| | | | | | | | | | | N | N | | | | | | N | |
| | | | | | | | | | | Q | Q | | | | | | Q | |
| | | | | | | | | | | S | S | | | | | | S | |
| | | | | | | | | | | T | T | | | | | | T | |
| | 1 substitutions; total epitope score = 17 | | | | | | | | | | | | | | | | | |
| variant | I | E | W | L | K | N | G | G | P | S | S | G | A | P | P | P | S | C |
| epitope | 0 | 0 | 4 | 3 | 0 | 0 | 0 | 0 | 0 | 0 | 0 | 0 | 0 | 0 | 0 | 0 | 0 | 0 |
| scores | | | | | | | | | | | | | | | | | | |
| variant | I | E | W | L | K | N | G | G | P | S | S | G | A | P | P | P | S | C |
| epitope | 0 | 0 | 4 | 3 | 0 | 0 | 0 | 0 | 0 | 0 | 0 | 0 | 0 | 0 | 0 | 0 | 0 | 0 |
| scores | | | | | | | | | | | | | | | | | | |

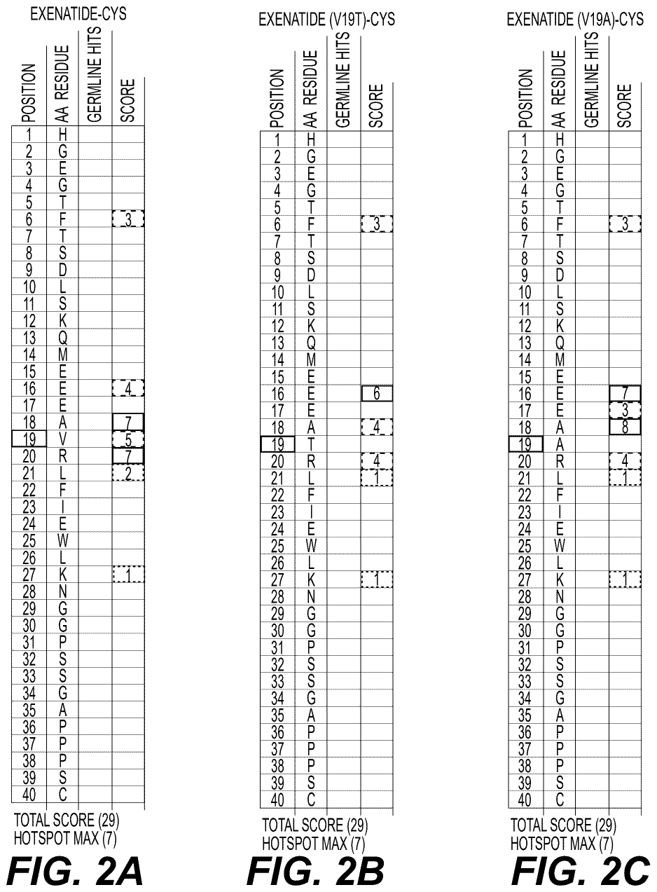

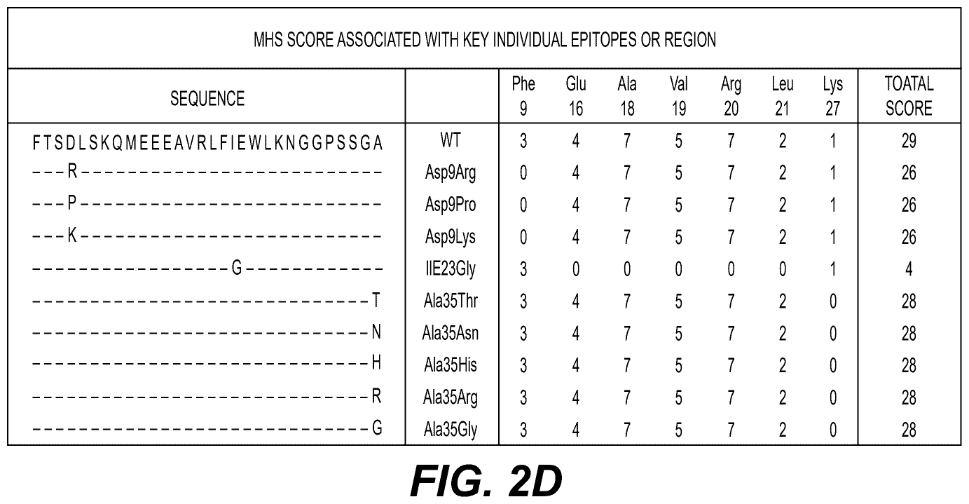

The V19 was confirmed as immunogenicity hot spot with a proprietary algorithm, iTope-AI, which predicts peptide binding specificities across 46 major HLA-DR, DP and DQ Isotypes, FIGS. 2A-2C. The 46 selected isotypes represent the most common HLA alleles found world-wide with no weighting attributed to those found most prevalently in any ethnic population. The location of key binding residues is achieved by the in-silico generation of 9 mer peptides that overlap by eight amino acids spanning the test protein sequence. Assessment of iTope-AI performance showed that the algorithm was able to accurately predict 95% of peptide binding core motifs previously identified by 3D structure from X-ray crystallography. Known HLA-DR, DP and DQ promiscuous binders were also predicted to bind the expected isotypes, demonstrating iTope-AI specificity.

This algorithm assigned Exenatide-Cys, Exenatide (V19T)-Cys and Exenatide (V19A)-Cys scores of 29, 19, and 27, respectively. None of the peptide sequences matched T cell epitopes that were identified in T Cell Epitope Database (TCED) (Bryson C J, Jones T D, Baker M P. BioDrugs. 2010 Feb. 1; 24(1): 1-8).

iTope-AI also identified acid substitutions that were predicted to have reduced MHC Class II binding at positions 9, 23, and 35. Initial identification and confirmation of Valine 19 as hot spot was carried out using multivariant analysis of the three sequences. Each row represents the position of the P1 anchor residue of a 9 mer peptide which is scored for MHC class II binding to all 46 HAL-DR, DP and DQ alleles in the right-hand side column of FIGS. 2A-2C. A ‘Position Risk Score’ of 1-2, 3-5, 6+ is highlighted in light, medium or dark grey, respectively. ‘Total Score’ corresponds to the sum of all ‘Position Risk Scores’ for the whole sequence and ‘Hotspot Max’ to the single highest ‘Position Risk Score’. Amino acid substitutions at position 9, 23 and 35 showed reduced predicted MHC Class II binding, as depicted in FIG. 2D.

### Ex Vivo T-Cell Proliferation Analysis of Deimmunized Pegylated Exenatide Variants

In silico prediction of T-Cell epitopes provides the initial step to assess immunogenicity potential, it does not account for all factors that influence clinical immunogenicity such as antigen processing, T cell receptor recognition of the MHC class II/peptide complex, T cell tolerance to any non-germline peptide, protein structure, formulation, pharmacology, and patient history, etc. For this reason, any analysis of sequences using predictive MHC class II binding tools may result in an over-prediction of antigen presentation and/or T cell epitopes. On the other hand, although predictive algorithms have been greatly improved, under-prediction of antigen presentation (i.e., false negatives) cannot be fully excluded.

To further assess the immunogenic potential of these peptides, the immunogenic potential of pegylated exenatide variants was tested in an ex vivo T-cell proliferation assay. A cohort of 50 donors was selected to best represent the number and frequency of HLA-DR and HLA-DQ allotypes expressed in European/North American and the world population. HLA-DP is not considered in the selection, due to its more limited polymorphism and likely low levels of expression. Bulk cultures were established using CD8+ T cell depleted PBMCs, and CD4+ T cell proliferation was measured at various time points after the addition of the sample by incorporation of [3H]-Thymidine. For proliferation (n=3 per time point), positive responses were defined by statistical and empirical thresholds as follows:
1. Significance (p<0.05) of the response by comparing CPM of test wells against medium control wells using unpaired two sample Student's t-test.
2. SI≥1.90, where SI=mean of test wells (CPM)/baseline (CPM).

For proliferation analysis, donors that were positive on at least one time point during the time course were deemed positive donors.

As shown in Table 2, the T-cell proliferation responses induced by all exenatide variants, regardless of amino acid sequence variation, the presence of PEG and the structure of the PEG, were comparable to HERCEPTIN®, the low immunogenicity control, indicating a low risk of immunogenicity. The magnitude and response rate for all PEGylated exenatide variants was lower than Bydureon (positive clinical benchmark control).

TABLE 2
T-Cell proliferation response induced by exenatide variants.

| Sample ID | Mean SI | SD | % Response |
|---|---|---|---|
| Exenatide-Cys-NPEG (NLY01) | 2.78 | ±0.80 | 6 |
| Exenatide-Cys (NLY01 peptide | 2.28 | ±0.32 | 4 |
| intermediate) | | | |
| Exenatide-Cys-JPEG | 2.31 | ±0.04 | 4 |
| Exenatide(V19T)-Cys-JPEG | 2.34 | ±0.50 | 6 |
| Exenatide(V19A)-Cys-JPEG | 2.54 | ±0.78 | 4 |
| Exenatide(I23G)-Cys-JPEG | N/A | N/A | 0 |
| Exenatide(D9P/123G)-Cys-JPEG | 2.23 | ±0.36 | 4 |
| Herceptin | 2.88 | ±0.62 | 8 |
| Bydureon | 2.79 | ±0.71 | 20 |
| KLH | 5.71 | ±4.83 | 84 |

### Potency of Deimmunized Pegylated Exenatide Variants

The bioactivity of the NLY01 with different PEG sources and deimmunized pegylated exenatide variants were compared in cell-based CAMP potency assay. In this assay, GLP-1R agonist activate the adenylyl cyclase pathway resulting in increased intracellular levels of cAMP. Stable GLP-1R expressing HEK293 cells co-transfected with a luciferase gene controlled by a cAMP responsive element CRE-luc were used in this potency assay. Expression level of the luciferase gene is directly proportional to the intracellular cAMP produced upon stimulation of the GLP1-R by NLY01 and deimmunized pegylated exenatide variants. Luminescence was measured using One-Glo™ luciferase assay system. The biological activity of NLY01 and deimmunized pegylated exenatide variants is expressed as the effective concentration inducing 50% stimulation of the maximum response (EC50).

As indicated in Table 3, both NLY01 with two different PEG sources (Exenatide-Cys-NPEG50K and Exenatide-Cys-JPEG50K) showed bioactivity with comparable EC50s. The V19 variants had similar but slightly lower EC50. However, the I23G and D9P/I23G variants had no significant bioactivity. Based on these results and the T-cell proliferation data, Exenatide (V19T)-Cys-JPEG50K and Exenatide (V19A)-Cys-JPEG50K were selected as the two most promising deimmunized pegylated exenatide.

TABLE 3
Cell-based potency of deimmunized pegylated exenatide variants

| Exenatide Variant | EC50, pM |
|---|---|
| Exenatide-Cys-NPEG50K (NLY01) | 40.3 |
| Exenatide-Cys-JPEG50K | 43.4 |
| Exenatide(V19T)-Cys-JPEG50K | 69.4 |
| Exenatide(V19A)-Cys-JPEG50K | 87.5 |
| Exenatide(I23G)-Cys-JPEG50K | 17740 |
| Exenatide(D9P/123G)-Cys-JPEG50K | No activity |

### Example 2: Efficacy of Deimmunized Exenatide Variants in Mice

In Vivo Efficacy of Deimmunized Exenatide Variants by Intraperitoneal Glucose Tolerance Test (ipGTT) in C57BL/6, Male Mice

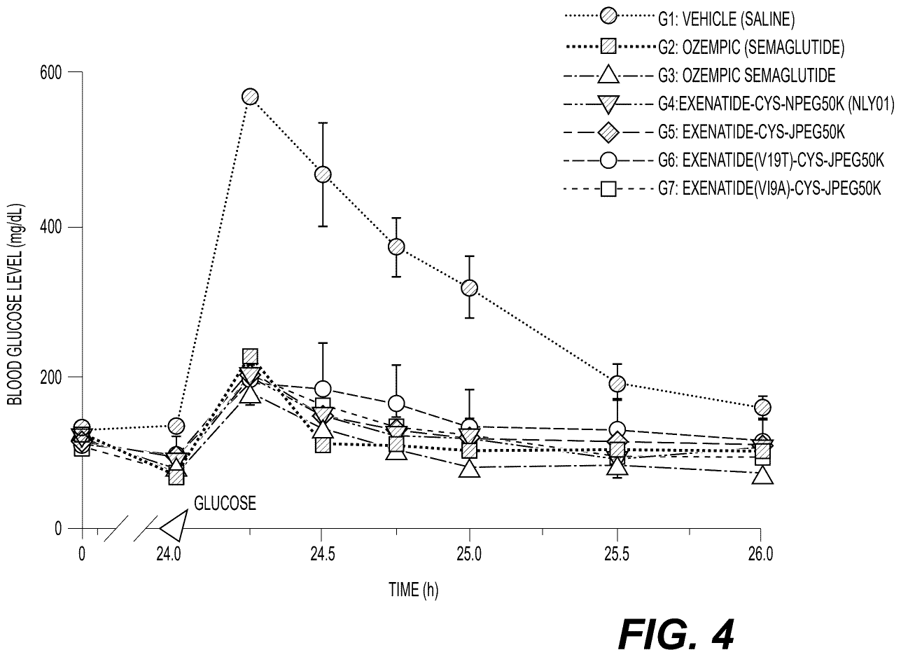

The in vivo efficacy of the NLY01 with different PEG sources and deimmunized pegylated exenatide variants was compared in C57BL/6, male mice by intraperitoneal glucose tolerance test (ipGTT). The mice were dosed with NLY01 variants including different PEGs and peptide variations. Semaglutide was included as an active comparator. Study scheme was shown in FIG. 3 and Table 3A. ipGTT tests were conducted at 24, 48, and 72 hours after a single subcutaneous dose. The ipGTT data 24 hours after dosing was shown in FIG. 4. NLY01 with two different PEG sources (Exenatide-Cys-NPEG50K and Exenatide-Cys-JPEG50K) and the V19 variants, Exenatide (V19T)-Cys-JPEG50K and Exenatide (V19A)-Cys-JPEG50, all had similar in vivo efficacy, which was comparable to semaglutide.

TABLE 3A
In-vivo efficacy study (ipGTT)

| | | | Injection | | | Dosing | | | |
|---|---|---|---|---|---|---|---|---|---|
| Group | Dose | | Volume | Route | N | Interval | Dosing | Glucose | Glucose Measurement |
| G1: Vehicle (Saline) | — | | 10 mL/kg | SC | 3 | single | −24 hour | 2 g/kg, IP | −24 hr (before TA |
| G2: Ozempic | 16.7 nmol/kg | Same nmole to | 10 mL/kg | SC | 4 | single | −24 hour | 2 g/kg, IP | dosing), |
| (semaglutide) | (66.8 ug/kg) | NLY01 | | | | | | | 0 (before glucose |
| G3: Ozempic | 50 nmol/kg | HED of 1 mg | 10 mL/kg | SC | 4 | single | −24 hour | 2 g/kg, IP | injection), |
| (semaglutide) | (0.2 mg/kg) | | | | | | | | 15, 30, 45, 60, 90, 120 |
| G4: Exenatide-Cys- | 16.7 nmol/kg | HED of 5 mg | 10 mL/kg | SC | 4 | single | −24 hour | 2 g/kg, IP | min |
| NPEG50K (NILY01) | (1 mg/kg) | | | | | | | | |
| G5: Exenatide-Cys- | 16.7 nmol/kg | | 10 mL/kg | SC | 5 | single | −24 hour | 2 g/kg, IP | |
| JPEG50K | (1 mg/kg) | | | | | | | | |
| G6: Exenatide (V19T)- | 16.7 nmol/kg | | 10 mL/kg | SC | 5 | single | −24 hour | 2 g/kg, IP | |
| Cys-JPEG50K | (1 mg/kg) | | | | | | | | |
| G7: Exenatide (V19A)- | 16.7 nmol/kg | | 10 mL/kg | SC | 5 | single | −24 hour | 2 g/kg, IP | |
| Cys-JPEG50K | (1 mg/kg) | | | | | | | | |

Glycemic Control in db/db Mice—Single Dose Study

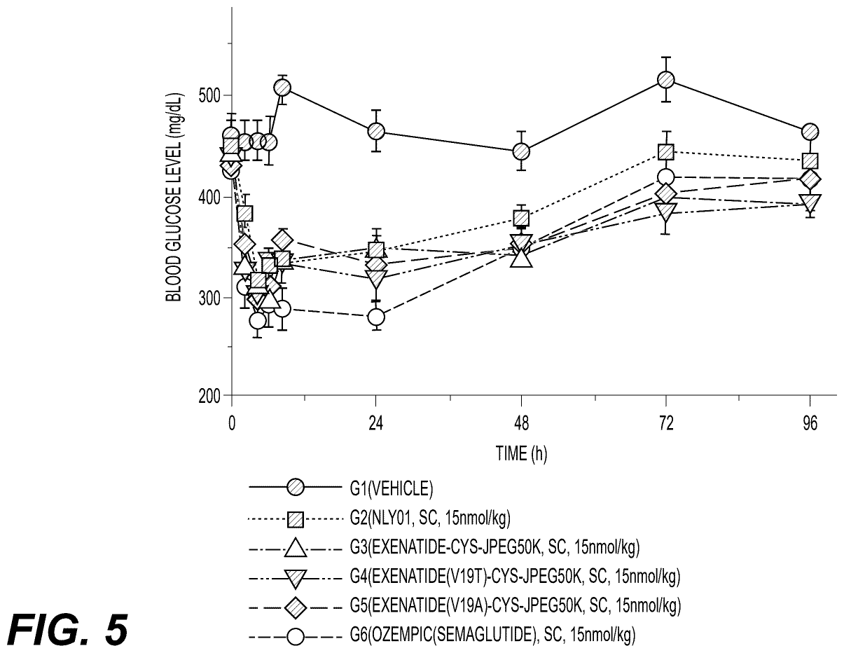

The in vivo efficacy of the NLY01 with different PEG sources and deimmunized pegylated exenatide variants was further examined in a diabetic mouse model. db/db mice were dosed with variants including different PEG sources, deimmunized pegylated exenatide variants, and semaglutide comparator. After a single dose, fed blood glucose was measured at 0 through 96 hours (Table 4). As shown in FIG. 5, both NLY01 with two different PEG sources (Exenatide-Cys-NPEG50K and Exenatide-Cys-JPEG50K) and the V19 deimmunized pegylated exenatide variants, Exenatide (V19T)-Cys-JPEG50K and Exenatide (V19A)-Cys-JPEG50, provided similar reductions in blood glucose.

TABLE 4
In vivo efficacy study of the NLY01 with different PEG sources and
deimmunized pegylated exenatide variants in diabetic mice model

| | | | | | Dosing | |
|---|---|---|---|---|---|---|
| Group | TA | Dose | Route | N | interval | Measurement |
| G1 | Vehicle | | SC | 5 | single | Fed BG (0, 2, |
| G2 | Exenatide-Cys-JPEG50K | 15 nmol/kg | SC | 5 | single | 4, 6,8, 24, 48, |
| | (NLY01) | | | | | 72, 96 h) |
| G3 | Exenatide-Cys-JPEG50K | 15 nmol/kg | SC | 5 | single | |
| G4 | Exenatide(V19T)-Cys- | 15 nmol/kg | SC | 5 | single | |
| | JPEG50K | | | | | |
| G5 | Exenatide(V19A)-Cys- | 15 nmol/kg | SC | 5 | single | |
| | JPEG50K | | | | | |
| G6 | Ozempic (semaglutide) | 15 nmol/kg | SC | 5 | single | |

Glycemic Control in db/db Mice-Repeated Dose Study

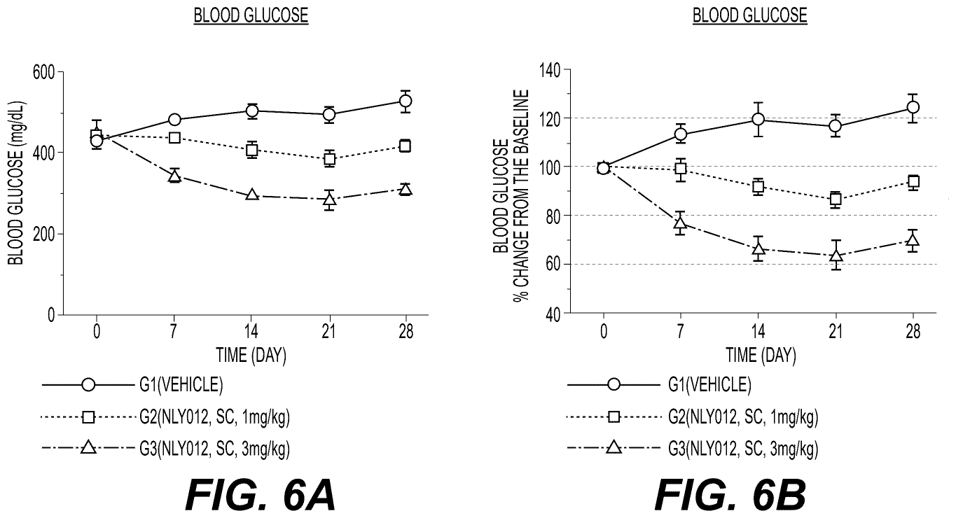

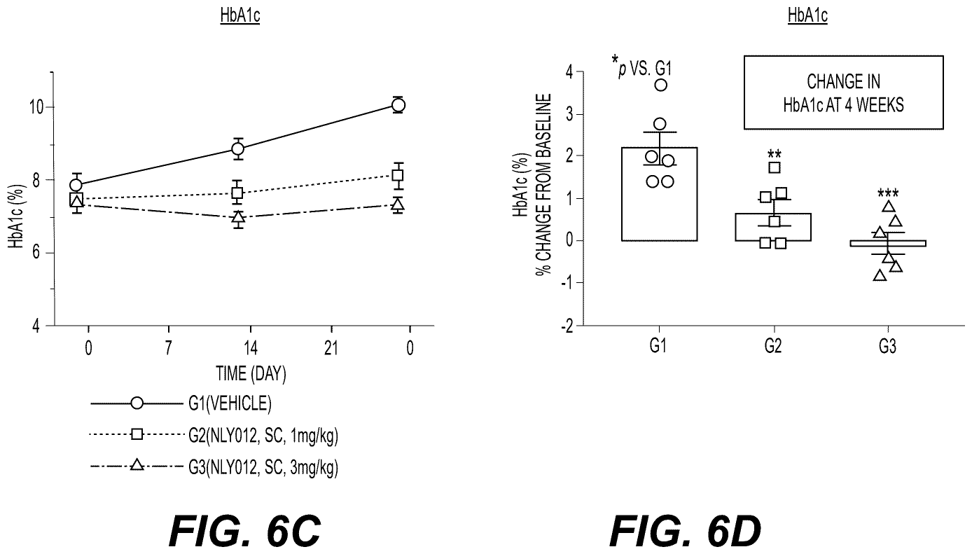

The in vivo efficacy of repeated administration of NLY12, i.e., Exenatide (V19A)-Cys-JPEG50, was further examined in diabetic db/db mice model. Dosing was twice weekly on Days 0, 3, 7, 10, 14, 17, 21, and 24 at 1 mg/kg and 3 mg/kg (Table 5). Fasting blood glucose was measured weekly, HbA1c was measure on Days −1, 13 and 27, and ipGTT was evaluated on Days-1 and 27. As shown in FIGS. 6A-6D, Exenatide (V19A)-Cys-JPEG50 (also referred to as NLY12) administration resulted in a dose-dependent reduction in fasting blood glucose and HbA1c levels.

TABLE 5
In vivo efficacy repeated dose study of Exenatide(V19A)-
Cys-JPEG50 (NLY12) in diabetic mice model

| | | | Dosing | | | Dosing |
|---|---|---|---|---|---|---|
| Group | TA | Dose | Volume | Route | N | interval |
| G1 | Vehicle | — | 5 mL/kg | SC | 6 | BIW |
| G2 | NLY12 | 1 mg/kg | 5 mL/kg | SC | 6 | BIW |
| G3 | NLY12 | 3 mg/kg | 5 mL/kg | SC | 6 | BIW |

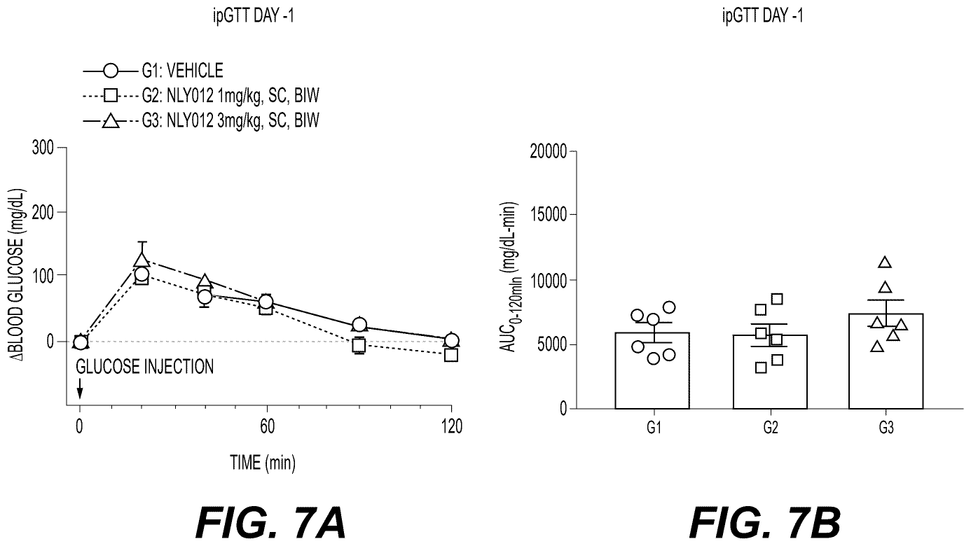

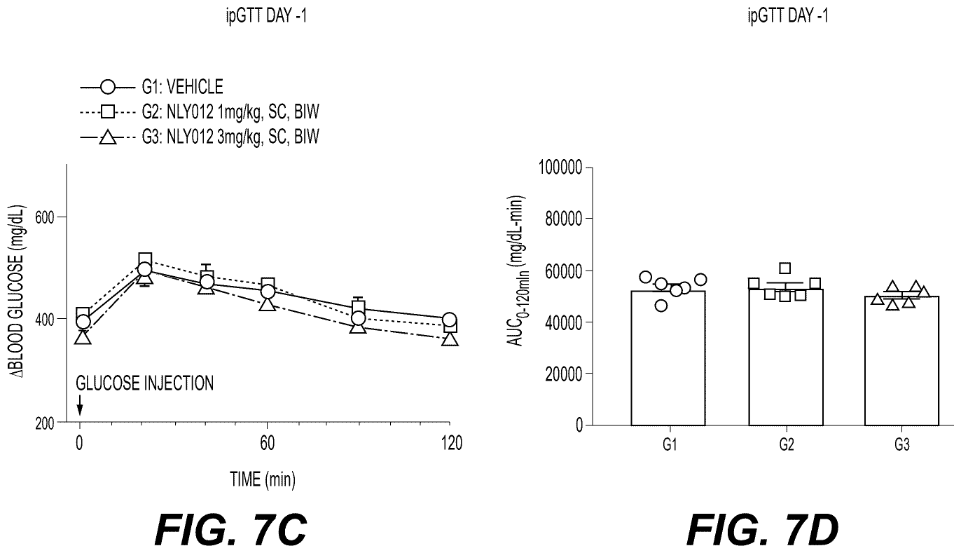

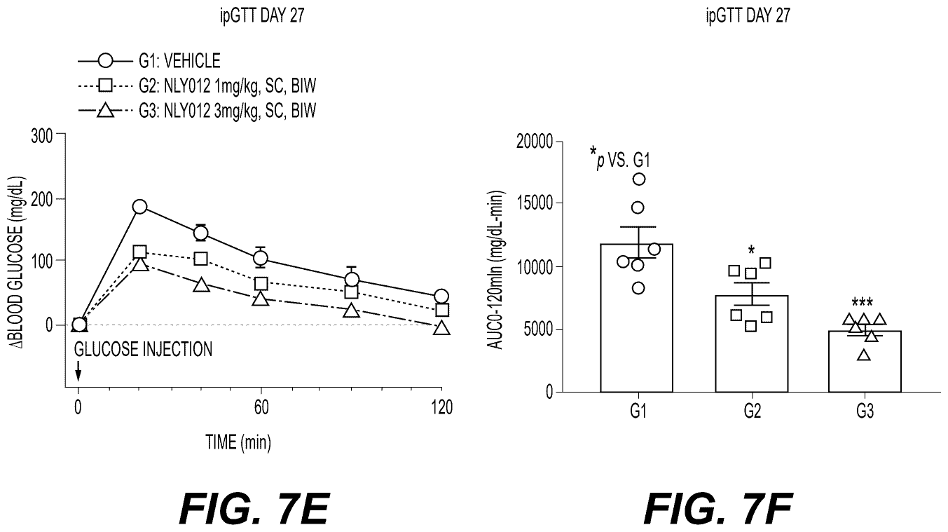

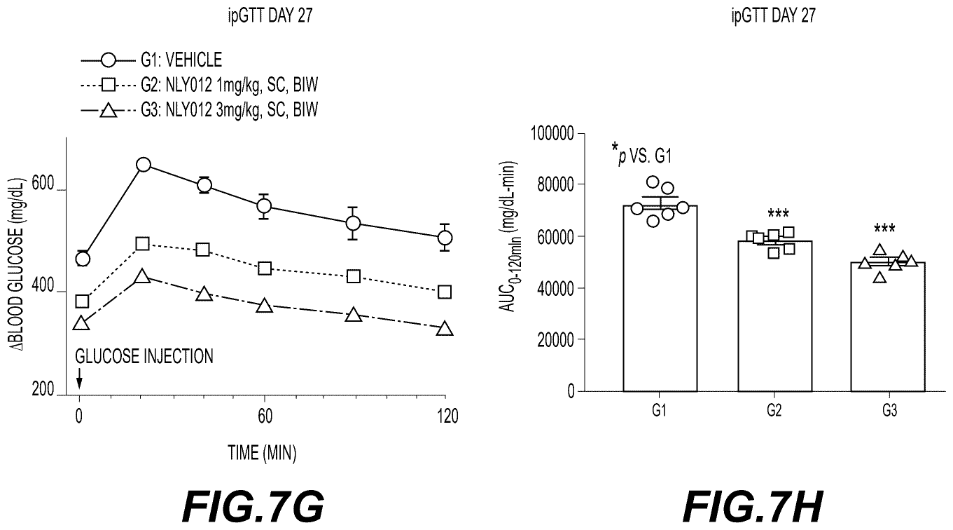

In addition, ipGTT results demonstrated that the glucose tolerance was significantly improved in Exenatide (V19A)-Cys-JPEG50 (NLY12) treated animals on Day 27, compared to controls (in FIGS. 7A-7H).

### Pharmacokinetic Profile Comparison in Non-Human Primate

The pharmacokinetic (PK) profile of NLY01 PEG and peptide variants, was assessed in cynomolgus monkeys. Two male and one female monkeys per treatment group were given NLY01 or variants at 7 mg/kg SC. Blood was collected pre-dose, then 1, 4, 8, 24, 48, 72, 120, and 168 hours after dosing, then again on Days 15 and Day 22 for pharmacokinetic analysis (Table 6).

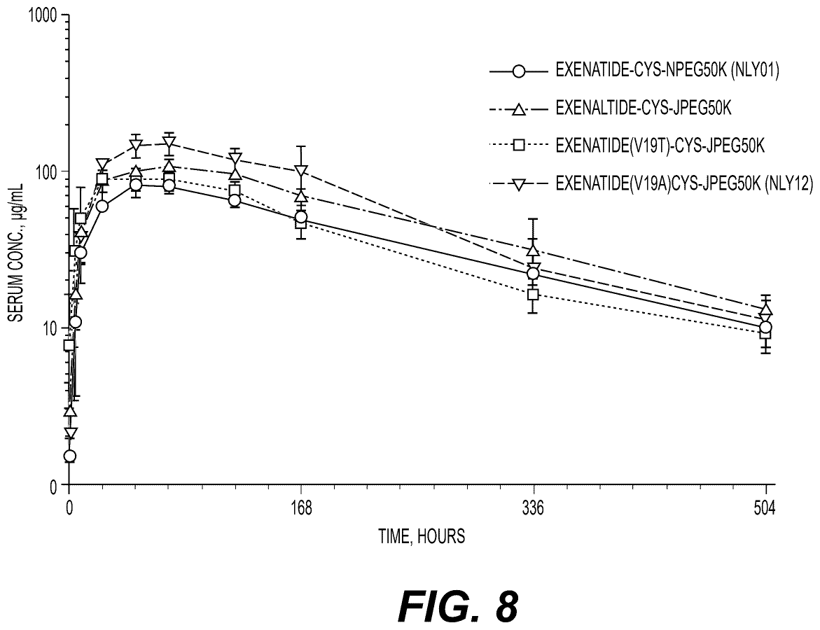

A single SC dose of 7 mg/kg was well-tolerated for all variants in cynomolgus monkeys. There were no treatment-related effects on body weight, hematology, coagulation, or serum chemistry parameters. Overall, the PK profiles of all variants tested were very similar. Time-concentration profiles are shown in FIG. 8 and key pharmacokinetic parameters are provided in Table 7.

TABLE 6
Pharmacokinetic comparison study of deimmunized
pegylated exenatide variants and NLY01 with
different PEG sources in non-human primate

| | Number of | | | Dose | Dose |
|---|---|---|---|---|---|
| Dose | Animals | | | Level | Volume |
| Group | (M/F) | Test Article | Route | (mg/kg) | (mL/kg) |
| 1 | 2/1 | Exenatide-Cys-JPEG50K | SC | 7 | 1 |
| | | (NLY01) | | | |
| 2 | 2/1 | Exenatide-Cys-JPEG50K | SC | 7 | 1 |
| 3 | 2/1 | Exenatide(V19T)-CYS- | SC | 7 | 1 |
| | | JPEG50K | | | |
| 4 | 2/1 | Exenatide(V19A)-CYS- | SC | 7 | 1 |
| | | JPEG50K | | | |

TABLE 7
Key Pharmacokinetic parameters for NLY01 with different PEG sources (Exenatide-
Cys-NPEG50K and Exenatide-Cys-JPEG50K) and the V19 deimmunized pegylated exenatide
variants, Exenatide(V19T)-Cys-JPEG50K and Exenatide(V19A)-Cys-JPEG50

| | Cmax | AUC 0-t | AUC 0-info_obs | T½ |
|---|---|---|---|---|
| Test Article | (μg/mL) | (μg/ml*h) | (μg/ml*h) | (days) |
| NLY01 | 83.1 ± 8.2 | 19542.3 ± 2094 | 21602.4 ± 2632 | 5.9 ± 0.5 |
| Exenatide-Cys-JPEG50K | 110.2 ± 9.8 | 27172.7 ± 2119 | 29864.5 ± 2916 | 5.8 ± 0.5 |
| Exenatide(V19T)-Cys-JPEG50K | 95.8 ± 21.6 | 20055.5 ± 4131 | 21845.9 ± 4368 | 5.6 ± 0.6 |
| Exenatide(V19A)-Cys-JPEG50K | 156 ± 25 | 35657 ± 9535 | 37353 ± 10098 | 4.4 ± 0.3 |

### Efficacy Study in Obese/T2D Non-Human Primates

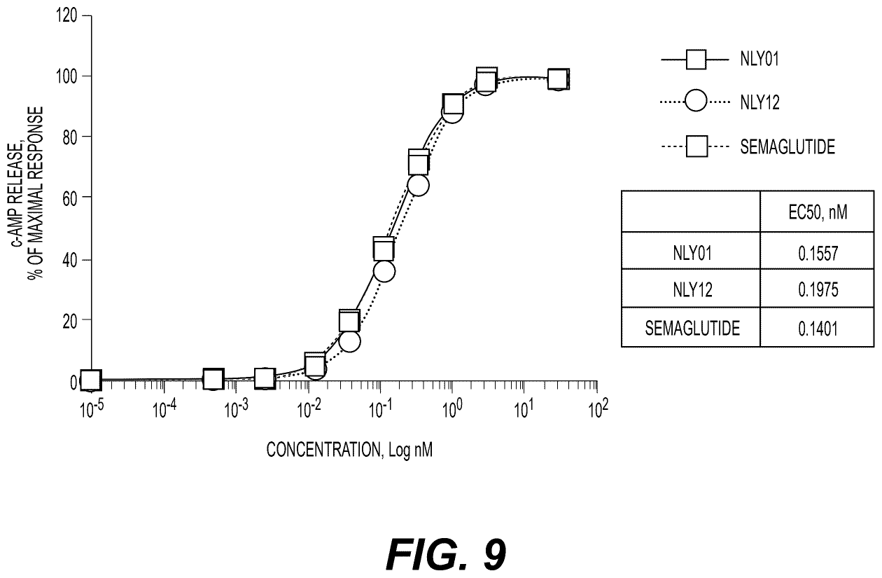

To further evaluate the potential of NLY12 (i.e., Exenatide (V19T)-Cys-JPEG50K) for clinical development, the potency of clinical scale lots of NLY01 and NLY12 were compared in a GLP-1R cAMP potency assay. As shown in FIG. 9, the concentration response curves for NLY12, NLY01, and for semaglutide are nearly superimposable with minimal difference in potency.

The effect of NLY12 was evaluated in obese diabetic male cynomolgus monkeys. These monkeys develop type 2 diabetes and obesity naturally through age and diet, providing a non-human primate model for disease. This study was designed to evaluate safety and tolerability of NLY12 following weekly or biweekly subcutaneous injection, and to evaluate the effect on fasting glucose, glucose tolerance, body weight, and HbA1c. Other parameters measured included food intake and clinical chemistry. One group of control animals (n=6) received only vehicle treatment (weekly). NLY12 was administered subcutaneously (SC) either weekly (7 doses on Days 1, 8, 15, 22, 29, 36 and 43) or biweekly (4 doses on Days 1, 15, 29 and 43) at 3 mg/kg (n=8/group) (Table 8).

TABLE 8
Efficacy study design of NLY12 in Obese/T2D NHPs.

| | | | Dose | | Dose | |
|---|---|---|---|---|---|---|
| | Number of | | Level | Concentration | Volume | Dose |
| Group | Animals | Treatment | (mg/kg) | (mg/mL) | (mL/kg) | Frequency |
| 1/A | 6 | Vehicle | — | — | 1 | Weekly ×7 |
| 2/B | 8 | NLY12 | 3 | 3 | 1 | Weekly ×7 |
| 3/C | 8 | NLY12 | 3 | 3 | 1 | Biweekly ×4 |

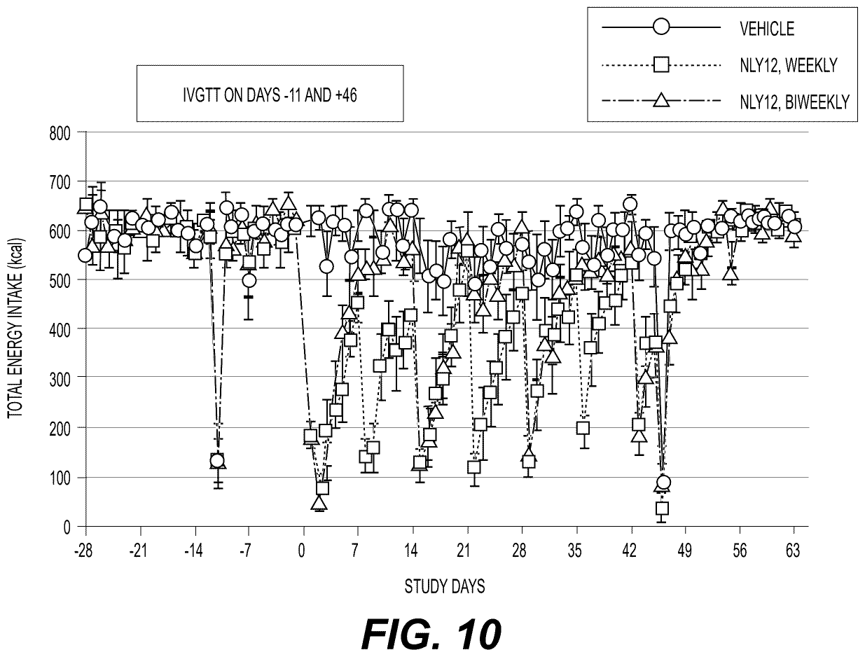

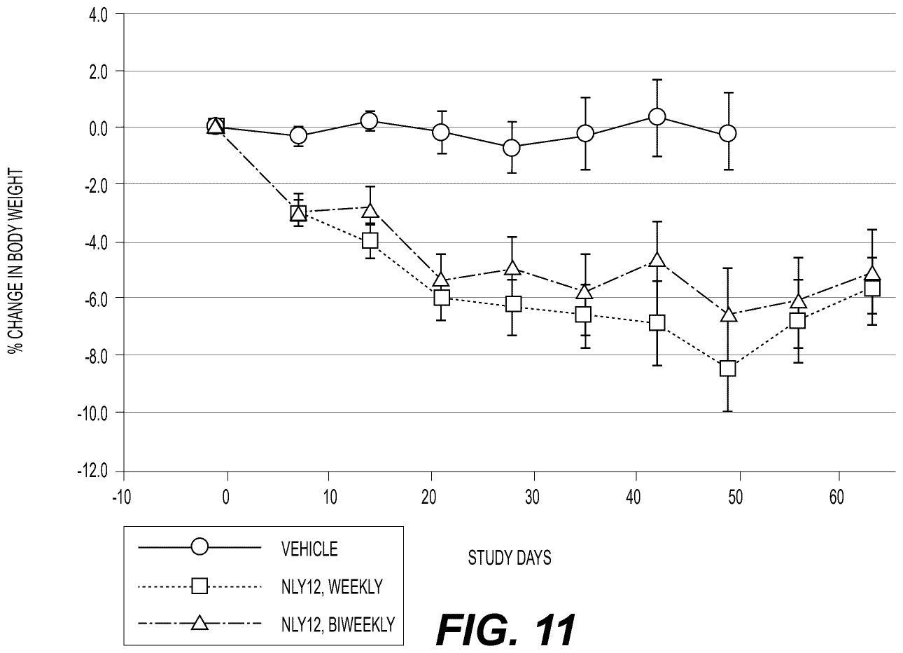

Food intake was significantly lower in the NLY12-treated groups compared to controls. Throughout the dosing period, reduced food consumption was observed in NLY12-treated animals starting from the day of each dose administration followed by gradual recovery within 3-5 days post dose (FIG. 10). NLY12 treatment resulted in significant body weight reduction starting from one week post dose. While body weights were stable in the control animals, weekly and bi-weekly administration of NLY12 resulted in mean weight reductions of 8.4% and 6.6%, respectively, on day 49 (compared to pre-dose baseline), FIG. 11.

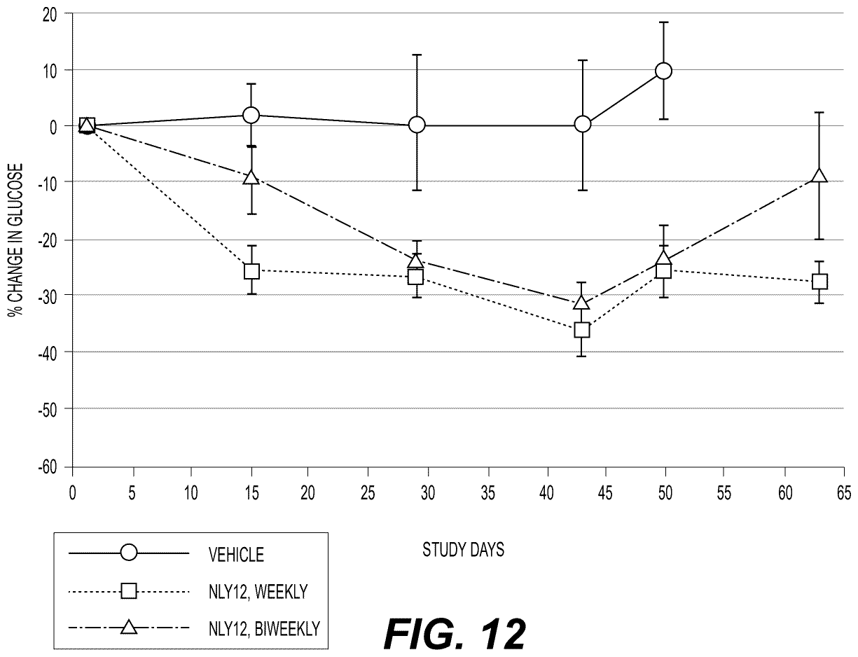

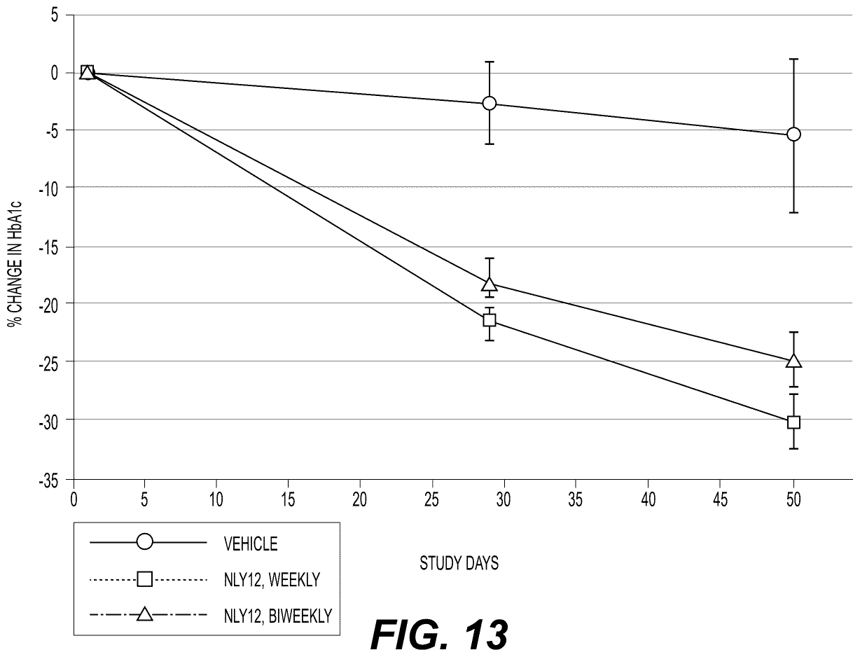

Fasting blood glucose and HbA1c levels were significantly decreased in NLY12 treated animals. Compared to baseline values, the mean blood glucose level was decreased by between 25% and 36%, and between 9% and 31% in weekly and bi-weekly NLY12 dosed animals, respectively (FIG. 12). The group mean HbA1c level was approximately 30% and 25% lower on Day 50 (compared to baseline) in weekly and bi-weekly NLY12 dosed groups, respectively (FIG. 13).

Intravenous glucose tolerance test (IVGTT) results indicated significant improvement in glucose tolerance following NLY12 administration. Plasma glucose at the end of the IVGTT (60 min) was approximately 55% lower and the group mean glucose AUC was 30-36% lower in NLY12 treated animals on Day 46 compared to baseline (Table 9). In addition, insulin AUC was moderately increased in treated animals on Day 46 (compared to baseline).

TABLE 9
Intravenous glucose tolerance test in Obese/T2D
NHPs treated with NLY12 at the end of IVGTT
(T = 60 min) Data are mean ± SEM;

| | | Plasma Glucose (mg/dL) | | |
|---|---|---|---|---|
| | | IVGTT @T = 60 min | | % Change |
| | Animal | Day −11 | Day +46 | from Baseline |
| Group | Numbers | (baseline) | (treatment) | Mean + SEM |
| Vehicle | 6 | 204 ± 22 | 173 ± 27 | −17.3 ± 7.0 |
| NLY12, weekly | 8 | 247 ± 18 | 106 ± 14 | −57.7 ± 3.7* |
| NLY 12, | 8 | 263 ± 25 | 123 ± 21 | −54.5 ± 6.1* |
| biweekly | | | | |

*p < 0.05 vs. Vehicle

As shown in Table 10 and Table 11, after weekly subcutaneous administration, NLY12 was absorbed, with median Tₘₐₓ values of 120 hours on Day 1 and 60 hours on Day 43. NLY12 mean Cmax and AUC₀₋₁₆₈ values were generally higher on Day 43 as compared to Day 1, with mean accumulation ratio values for Cₘₐₓ and AUC0-168 of 2.7 and 3.1 respectively, on Day 43.

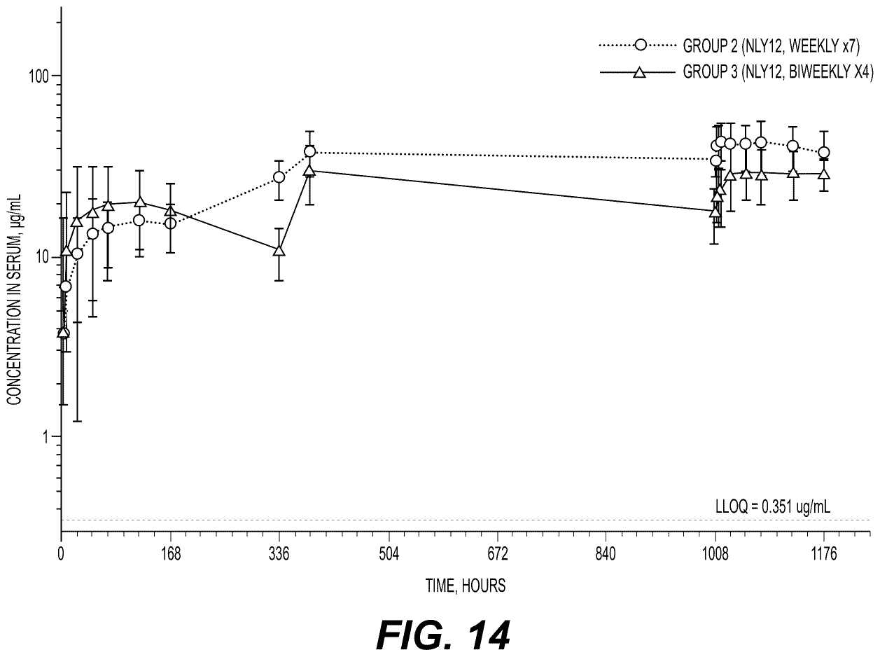

After bi-weekly subcutaneous administration, NLY12 was absorbed, with median Tₘₐₓ values of 120 hours on Day 1 and 96 hours on Day 43. The elimination half-life (T_{1/2}) was approximately 10 days on Day 1. NLY12 mean Cₘₐₓ and AUC0-168 values were generally higher on Day 43 as compared to Day 1. Mean accumulation ratio values for Cₘₐₓ and AUC₀₋₁₆₈ were 1.5 and 1.6 respectively, on Day 43. Mean concentration values for NLY12 were measurable through the last scheduled time point (168 hours post-dose on Days 1 and 43), FIG. 14.

TABLE 10
NLY12 PK parameters in T2D/Obese monkeys following
Weekly SC administration at 3 mg/kg/dose.

| | Cₘₐₓ | AUC₀₋₁₆₈ |
|---|---|---|
| Day | (μg/mL) | (h*μg/mL) |
| 1 | 16.9 ± 6.7 | 2277.2 ± 894 |
| 43 | 46.3 ± 12.5 | 7006.4 ± 1957 |

| | | |
|---|---|---|
| Accumulation Ratio (D 43/D 1) | 2.7 | 3.1 |

| | | |
|---|---|---|
| Trough Conc. (μg/mL) | 15 | 27.6 ± 6.9 |
| | 43 | 34.8 ± 7.5 |

TABLE 11
NLY12 PK parameters in T2D/Obese monkeys following
bi-weekly SC administration at 3 mg/kg/dose.

| | Cmax | AUC0-168 |
|---|---|---|
| Day | (μg/mL) | (h*μg/mL) |
| 1 | 22.0 ± 13.3 | 3031.9 ± 1870.60 |
| 43 | 32.6 ± 10.6 | 4879.0 ± 1558.7 |

| | | |
|---|---|---|
| Accumulation Ratio (D 43/D 1): | 1.5 | 1.6 |

| | | |
|---|---|---|
| Trough Conc. (μg/mL) | 15 | 11.1 ± 3.6 μg/mL |
| | 43 | 18.3 ± 6.5 μg/mL |

In conclusion, subcutaneous administration of NLY12 either weekly or bi-weekly for 7-weeks at 3 mg/kg/dose was well-tolerated in obese/diabetic cynomolgus monkeys. NLY12 administration resulted in a significant decrease in food consumption and a body weight loss of approximately 6-8%. NLY12-treated monkeys had lower total cholesterol, low density lipoprotein cholesterol, and triglycerides, as well as significantly decreased fasting blood glucose and HbA1c levels. In an IVGTT, treated monkeys had significantly decreased glucose excursion, suggesting improved glucose tolerance. Overall, changes were more pronounced in monkeys treated weekly compared to bi-weekly treatment.

### Example 3: Modeled Pharmacokinetics of Exenatide (V19A)-Cys-JPEG50 (NLY12) Following Titration and Monthly Dosing

There is an earlier trial that illustrates the potential for a once-monthly GLP-1R agonist (Rosenstock J, Reusch J, Bush M, Yang F, Stewart M; Albiglutide Study Group. Diabetes Care. 2009 October; 32(10): 1880-6). This study was conducted in T2D to evaluate the effectiveness and tolerability of albiglutide (Tanzium) when administered weekly, bi-weekly, or monthly with doses adjusted to achieve similar exposure levels. In this randomized multicenter double-blind parallel-group study, 356 type 2 diabetic subjects with similar mean baseline characteristics received placebo, twice-daily exenatide (active control), or albiglutide weekly (4, 15, or 30 mg), biweekly (15, 30, or 50 mg), or monthly (50 or 100 mg) over a treatment period of 16 weeks. The primary efficacy outcome measure for the study was change from baseline HbA1c versus placebo at week 16.

At the end of treatment, mean HbA1c was similarly reduced from baseline at all dose intervals: 30 mg weekly, 50 mg biweekly, and 100 mg monthly (HbA1c reduction of 0.87, 0.79, and 0.87%, respectively) compared to placebo (HbA1c reduction of 0.17%, P<0.004). At these doses, weekly, bi-weekly, and monthly dosing appeared equally efficacious. Closer observation, however, revealed a substantial loss in fasting blood glucose control when albiglutide was administered monthly. This is explained by the PK data showing albiglutide concentrations fall essentially to zero between monthly doses. Monthly dosing was also associated with a significantly greater incidence of nausea and vomiting on dosing days than is observed at any time with weekly dosing. This may also be related to the fall in albiglutide concentrations below pharmacologically active levels as well. Continuous exposure to active levels may better promote GI tolerance, which is normally observed with repeat administration of GLP-1 RAs.

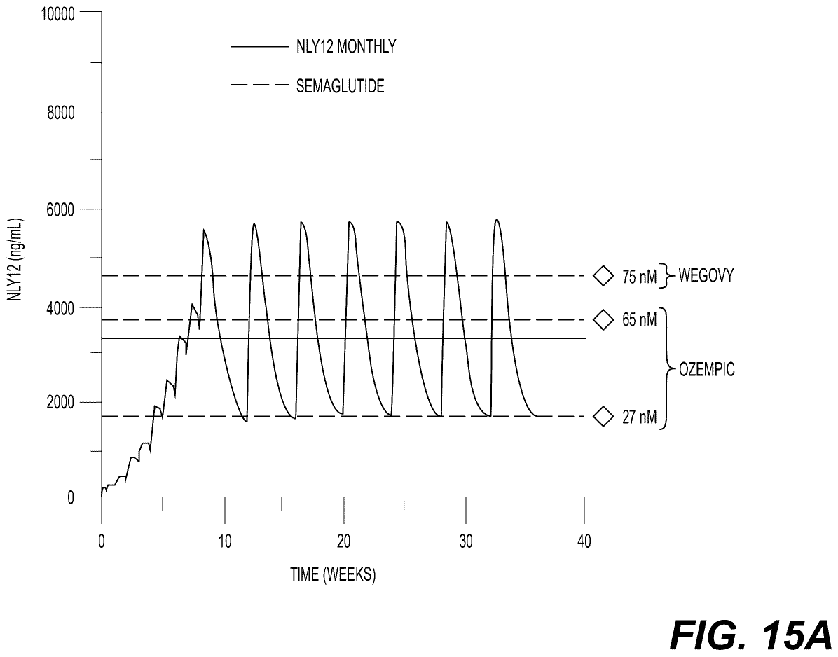

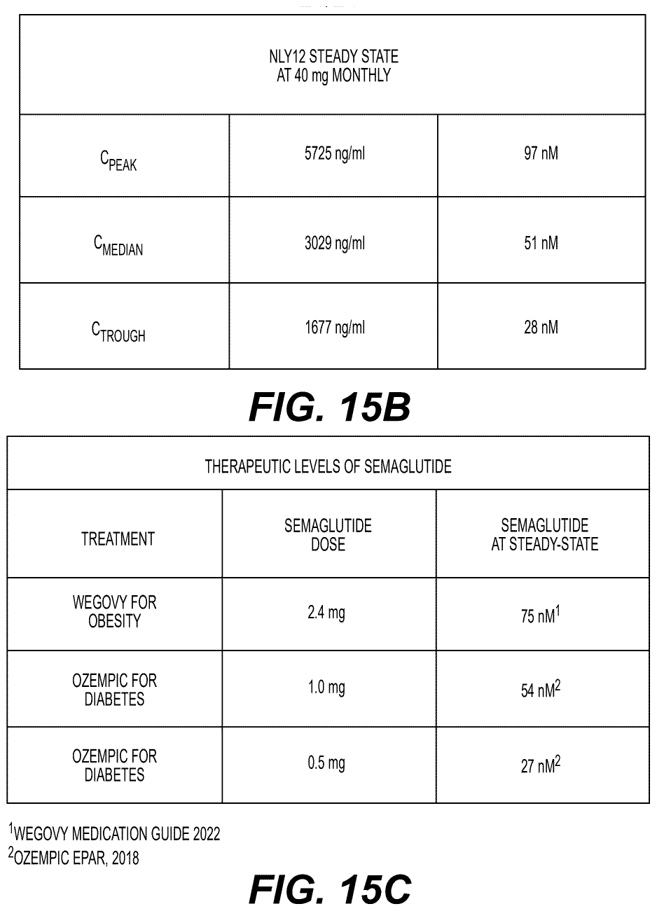

To evaluate the potential of NLY12 to provide once-monthly treatment for T2D and obesity, the PK of NLY12 in humans was modeled using a nonparametric superposition analysis modeled after NLY01 clinical PK data. In FIG. 15A, serum concentrations are modeled assuming an 8-week titration phase followed by monthly dosing. In this model, dosing during the titration phase is administered weekly at ascending doses of 2.5, 5, 10, and 15 mg, each given for 2 weeks before escalation. This titration phase is followed by a single 25 mg dose on week 9, allowing a smooth transition from titration to monthly dosing. The bridging dose on week 9 is followed by monthly doses of 40 mg. Estimated steady state concentrations for NLY12 are shown in FIG. 15B. Steady-state therapeutic levels of semaglutide (FIG. 15C) are aligned based on molar equivalence with NLY12.

Based on this model, NLY12 concentrations at steady state (peak, median, and trough) all overlap with known therapeutic concentration of semaglutide. Unlike was observed with albiglutide when administered monthly, the half-life of albiglutide is ~5 days in humans, whereas NLY01 variants have a half-life in the range of 12 days. With the half-life doubled, NLY12 remains at therapeutic levels throughout the dosing period. Continuous exposure at levels that remain efficacious may allow for the first once monthly GLP-1R agonist. Furthermore, titration to allow a gradual rise to steady state along with continuous exposure to pharmacologically active levels may reduce the GI side effects associated with dosing.

### SUMMARY

This study has identified a PEGylated exenatide analog suitable for once-monthly administration. The deimmunized exenatide peptides eliminate a major site of immunogenicity potential in the peptide (Table 12). De-immunization limits the potential for immune interference with treatment effectiveness and safety. Based on in silico algorithms, the 19th amino acid exenatide (valine, V19) was identified as an immunogenicity hot spot. A conservative substitution of the amino acid valine at 19 with alanine or threonine substantially reduced the predicted immunogenicity score. The resulting pegylated peptides, Exenatide (V19T)-Cys-JPEG50K and Exenatide (V19A)-Cys-JPEG50K, retained similar characteristics as the parent molecule and proved similar in activity to other PEGylated exenatide analogs in development (i.e., Exenatide-Cys-NPEG50K (NLY01) and Exenatide-Cys-JPEG50K, including:
Low immunogenicity potential as measured by response rate in T-cell proliferation assay (Table 2)
Potency in cell-based GLP-1R CAMP assay (Table 3, FIG. 9)
Glycemic control by ipGTT in C57BL/6, male mice (FIG. 4)
Glycemic control efficacy in db/db mice model (FIG. 5)
Pharmacokinetics (Cₘₐₓ, AUC, T½) in non-human primates (FIG. 8, Table 7)

Further, NLY12 (Exenatide (V19A)-Cys-JPEG50K) was validated as an important candidate through demonstration of the following:
dose-dependent reductions in fasting blood glucose, HbA1c level, and glucose tolerance in diabetic mice (FIGS. 6A-6D, 7A-7H)
reductions in body weight, fasting blood glucose, HbA1c level, and glucose tolerance in diabetic monkey (FIGS. 10-14; Tables 9-11).

In a comparison of Exenatide-Cys peptide conjugates with different 50 kD 3-arm, branched PEGs (JPEG50K and NPEG50K in FIGS. 1A and 1B), molecular characteristics were similar. Structural differences in these 3-arm branched 50 kD PEGs are minimal: 1) two additional carbons on the aliphatic chain of the 10 kD PEG arm and 2) different junction design at the 20 kD PEG branch arms. Differences in these PEG are not in regions thought to contribute to PEG immunogenicity. Recently it was shown that anti-PEG antibodies form an open ring structure, via Van der Waals interactions, that binds to PEG backbone repeats (—OCH₂CH₂—)ₙ (Huckaby, J. T., Jacobs, T. M., Li, Z. et al. Commun Chem 3, 124 (2020); Lee, CC., Su, YC., Ko, TP. et al. J Biomed Sci 27, 12 (2020)) and earlier studies suggested anti-PEG antibodies target the terminal end-groups of the PEGs (Kozma G T, Shimizu T, Ishida T, Szebeni J. Adv Drug Deliv Rev. 2020; 154-155:163-175).

Overall, the data demonstrate a major T-cell epitope in exenatide can be eliminated through a conservative substitution (V19A or V19T) while maintaining similar biological, pharmacological and pharmacokinetic properties.

TABLE 12
Exenatide variants

| | |
|---|---|
| Exenatide-Cys- | Pegylated peptide: His-Gly-Glu-Gly-Thr-Phe-Thr-Ser- |
| NPEG50K | Asp-Leu-Ser-Lys-Gln-Met-Glu-Glu-Glu-Ala-Val-Arg- |
| (NLY01) | Leu-Phe-Ile-Glu-Trp-Leu-Lys-Asn-Gly-Gly-Pro-Ser- |
| | Ser-Gly-Ala-Pro-Pro-Pro-Ser-Cys(NPEG50K)-NH₂ |
| | (SEQ ID NO: 8) |
| | Peptide intermediate: |
| | HGEGTFTSDLSKQMEEEAVRLFIEWLKNGGPSSG |
| | APPPSC (SEQ ID NO: 3) |
| | pI/Mw: 4.70/4290.75 by ExPASy |
| Exenatide-Cys- | Pegylated peptide: His-Gly-Glu-Gly-Thr-Phe-Thr-Ser- |
| JPEG50K | Asp-Leu-Ser-Lys-Gln-Met-Glu-Glu-Glu-Ala-Val-Arg- |
| | Leu-Phe-Ile-Glu-Trp-Leu-Lys-Asn-Gly-Gly-Pro-Ser- |
| | Ser-Gly-Ala-Pro-Pro-Pro-Ser-Cys(JPEG50K)-NH₂ |
| | (SEQ ID NO: 9) |
| | Peptide intermediate: |
| | HGEGTFTSDLSKQMEEEAVRLFIEWLKNGGPSSG |
| | APPPSC (SEQ ID NO: 3) |
| | pI/Mw: 4.70/4290.75 by ExPASy |
| Exenatide(V19T)- | Pegylated peptide: His-Gly-Glu-Gly-Thr-Phe-Thr-Ser- |
| Cys-JPEG50K | Asp-Leu-Ser-Lys-Gln-Met-Glu-Glu-Glu-Ala-Thr-Arg- |
| | Leu-Phe-Ile-Glu-Trp-Leu-Lys-Asn-Gly-Gly-Pro-Ser- |
| | Ser-Gly-Ala-Pro-Pro-Pro-Ser-Cys(JPEG50K)-NH₂ |
| | (SEQ ID NO: 10) |
| | Peptide intermediate: |
| | HGEGTFTSDLSKQMEEEATRLFIEWLKNGGPSSG |
| | APPPSC (SEQ ID NO: 4) |
| | pI/Mw: 4.70/4292.72 by ExPASy |
| Exenatide(V19A)- | Pegylated peptide: His-Gly-Glu-Gly-Thr-Phe-Thr-Ser- |
| Cys-JPEG50K | Asp-Leu-Ser-Lys-Gln-Met-Glu-Glu-Glu-Ala-Ala-Arg- |
| (NLY12) | Leu-Phe-Ile-Glu-Trp-Leu-Lys-Asn-Gly-Gly-Pro-Ser- |
| | Ser-Gly-Ala-Pro-Pro-Pro-Ser-Cys(JPEG50K)-NH₂ |
| | (SEQ ID NO: 11) |
| | Peptide intermediate: |
| | HGEGTFTSDLSKQMEEEAARLFIEWLKNGGPSSG |
| | APPPSC (SEQ ID NO: 5) |
| | pI/Mw: 4.70/4262.69 by ExPASy |
| Exenatide(I23G)- | Pegylated peptide: His-Gly-Glu-Gly-Thr-Phe-Thr- |
| Cys-JPEG50K | Ser-Asp-Leu-Ser-Lys-Gln-Met-Glu-Glu-Glu-Ala- |
| | Val-Arg-Leu-Phe-Gly-Glu-Trp-Leu-Lys-Asn-Gly- |
| | Gly-Pro-Ser-Ser-Gly-Ala-Pro-Pro-Pro-Ser- |
| | Cys(JPEG50K)-NH₂ (SEQ ID NO: 12) |
| | Peptide intermediate: |
| | HGEGTFTSDLSKQMEEEAVRLFGEWLKNGGPS |
| | SGAPPPSC (SEQ ID NO: 6) |
| | pI/Mw: 4.70/4234.64 by ExPASy |
| Exenatide(D9P/ | Pegylated peptide: His-Gly-Glu-Gly-Thr-Phe-Thr- |
| I23G)-Cys- | Ser-Pro-Leu-Ser-Lys-Gln-Met-Glu-Glu-Glu-Ala-Val- |
| JPEG50K | Arg-Leu-Phe-Gly-Glu-Trp-Leu-Lys-Asn-Gly-Gly- |
| | Pro-Ser-Ser-Gly-Ala-Pro-Pro-Pro-Ser- |
| | Cys(JPEG50K)-NH₂ (SEQ ID NO: 13) |
| | Peptide intermediate: |
| | HGEGTFTSPLSKQMEEEAVRLFGEWLKNGGPSS |
| | GAPPPSC (SEQ ID NO: 7) |
| | pI/Mw: 5.02/4216.67 by ExPASy |

Unless defined otherwise, all technical and scientific terms used herein have the same meanings as commonly understood by one of skill in the art to which the disclosed invention belongs. Publications cited herein and the materials for which they are cited are specifically incorporated by reference.

Those skilled in the art will recognize or be able to ascertain using no more than routine experimentation, many equivalents to the specific embodiments of the invention described herein. Such equivalents are intended to be encompassed by the following claims.

1. A Glucagon-like peptide-1 receptor agonist (GLP-1 RA) peptide or analog thereof having activities towards a GLP-1 receptor, comprising the amino acid sequence of
(SEQ ID NO: 1)
HGX₃GX₅X₆X₇X₈X₉X₁₀X₁₁X₁₂X₁₃X₁₄X₁₅X₁₆X₁₇X₁₈X₁₉X₂₀
X₂₁X₂₂X₂₃X₂₄WX₂₆X₂₇NGGPX₃₂X₃₃GX₃₅PPPX₃₉C,
wherein Xₙ is each independently selected from the amino acids for the specific positions:
X₃ is E, D, K, or Q;
X₅ is S or T;
X₆ is F or Y;
X₇ is S or T;
X₈ is S, or A, D, E, G, K, N, Q, or T;
X₉ is D, P, or E;
X₁₀ is L, F, I, M, or V;
X₁₁ is S, A, D, E, G, K, N, Q, or T;
X₁₂ is K, E, Q, or R;
X₁₃ is Q, E, K, or R;
X₁₄ is M, R, L, or V;
X₁₅ is E, D, K, or Q;
X₁₆ is E, D, K, or Q;
X₁₇ is E, D, K, or Q;
X₁₈ is A, G, S, T, or V;
X₁₉ is A, I, L, M, T, or V;
X₂₀ is R, K, or Q;
X₂₁ is L, F, I, M, or V;
X₂₂ is F or Y;
X₂₃ is I, G, L, F, M, or V;
X₂₄ is E, D, K, or Q;
X₂₆ is L, F, I, M, or V;
X₂₇ is K, E, Q, or R;
X₃₂ is S, A, D, E, G, K, N, Q, or T;
X₃₃ is S, A, D, E, G, K, N, Q, or T;
X₃₅ is A, G, S, T, or V; and
X₃₉ is S, A, D, E, G, K, N, Q, or T.

2. The GLP-1RA peptide or analog of claim 1, comprising the amino acid sequence of HGEGTFTSX₉LSKQMEEEAX₁₉RLFX₂₃EWLKNGGPSSGX₃₅PPPSC (SEQ ID NO: 2).

3. The GLP-1RA peptide or analog of claim 1, wherein X₁₉ is not a valine.

4. The GLP-IRA peptide or analog of claim 1, wherein X₁₉ is A or T.

5. The GLP-1RA peptide or analog of claim 1, wherein X₂₃ is G.

6. The GLP-1RA peptide or analog of claim 1, wherein X₉ is P and X₂₃ is G.

7. The GLP-1RA peptide or analog of claim 1, comprising the amino acid sequence of any one of SEQ ID NOs: 2-7.

8. The GLP-1RA peptide or analog of claim 1, comprising the amino acid sequence of SEQ ID NO: 5.

9. The GLP-1RA peptide or analog of claim 1, having a substantially lower immunogenicity in a mammalian host, preferably, human host, relative to immunogenicity of SEQ ID NO: 3.

10. The GLP-1RA peptide or analog of claim 1, wherein the GLP-1RA peptide or analog is conjugated to one or more polyethylene glycol (PEG) moieties.

11. The GLP-IRA peptide or analog of claim 1, wherein the peptide or analog is conjugated to one or more multiple-branched PEGs.

12. The GLP-1RA peptide or analog of claim 10, wherein the multiple-branched PEG has a molecular weight of between 20 kDa and 250 kDa.

13. The GLP-IRA peptide or analog of 12, wherein the multiple-branched polyethylene glycol is a 3-arm branched PEGs having a molecular weight of between 20 kDa and 250 kDa.

14. The GLP-1RA peptide or analog of claim 13, wherein the 3-arm branched PEGs having one of the following structures: 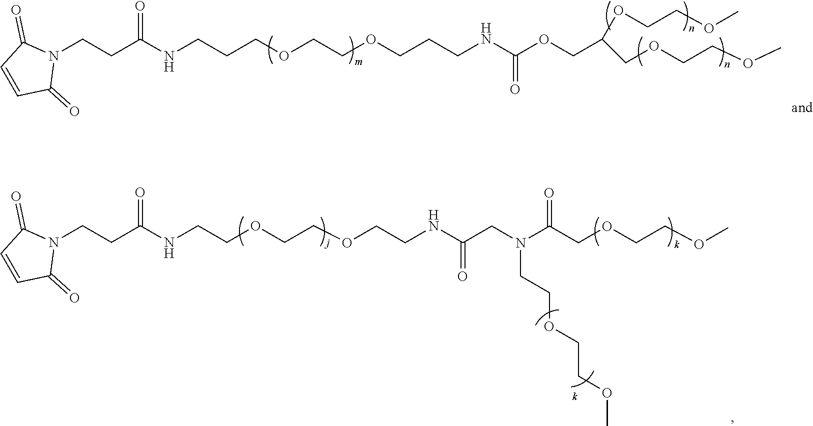
wherein m, n, j, k each is an integer independently between about 100 and 5,000.

15. The GLP-1RA peptide or analog of claim 11, wherein the multiple-branched polyethylene glycol is a 3-arm branched PEGs having a molecular weight of about 50 kDa.

16. The GLP-1RA peptide or analog of claim 11, wherein the peptide or analog conjugated to one or more polyethylene glycol (PEG) moieties comprises any one of SEQ ID NOs: 7-13.

17. The GLP-1RA peptide or analog of claim 1, wherein the GLP-1RA peptide or analog comprises the amino acid sequence of SEQ ID NO: 5, and conjugated via C-terminal cysteine residue a 3-arm branched PEGs having one of the following structures: 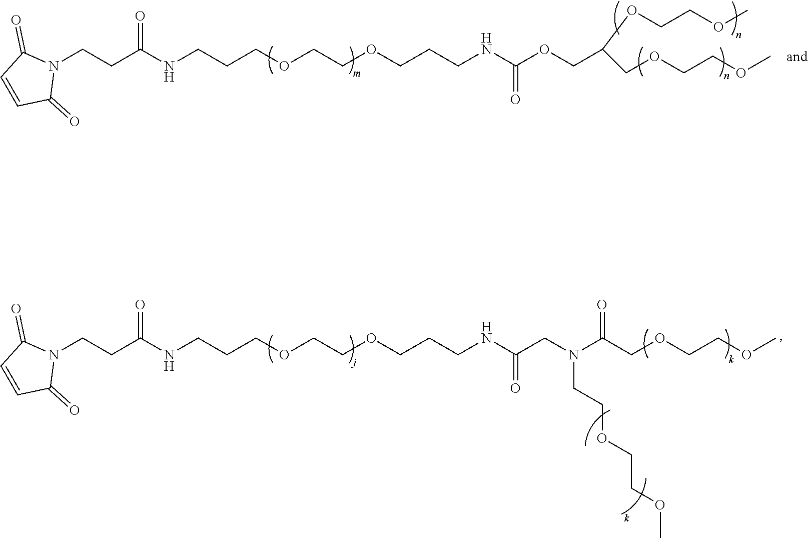
wherein m, n, j, k each is an integer independently between about 100 and 5,000, wherein the 3-arm branched PEG has a molecular weight of about 50 kDa.

18. A pharmaceutical formulation comprising the GLP-1RA peptide or analog of claim 1.

19. A method of treating one or more diseases selected from the group consisting of obesity, diabetes, and non-alcoholic fatty liver disease in a subject in need thereof, comprising administering to an effective amount of the pharmaceutical formulation of claim 18 to treat or alleviate one or more symptom of the one or more diseases.

20. The method of claim 19, wherein the pharmaceutical formulation is administered in an amount effective to induce weight loss, reduce the body fat, reduce food intake, improve glucose homeostasis, or combinations thereof, in a normal or obese patient.

21. The method of claim 19 or 20, wherein the pharmaceutical formulation is administered via a route selected from the group consisting of enteral administration and parenteral administration.

22. The method of claim 21, wherein the pharmaceutical formulation is administered via oral administration or subcutaneous administration.

23. The method of claim 21, wherein the pharmaceutical formulation is administered in a form selected from the group consisting of pills, capsules, tablets, liquids, and suspensions.

24. The method of claim 19, wherein the pharmaceutical formulation is administered at an interval of once a month, once every two weeks, once a week, or less frequent.

25. The method of claim 19, wherein the pharmaceutical formulation is administered the subject once a month.

26. The method of claim 19, wherein the pharmaceutical formulation is administered to the subject for a duration of between one and 10 year, inclusive.

27. The method of claim 19, wherein the pharmaceutical formulation is administered to a human subject at a dose of between 0.001 mg/kg body weight of the subject and 10 mg/kg body weight of the subject, inclusive.

28-29. (canceled)

30. The method of claim 19, wherein the pharmaceutical formulation provides a therapeutically effective concentration of the GLP-IRA peptide or analog in the serum of the subject throughout the treatment once a steady state is established after administration.

31. The method of claim 30, wherein the therapeutically effective concentration of the GLP-1RA peptide or analog in the serum of the subject during the treatment does not fall below pharmacologically active levels.

32. (canceled)

33. The method of claim 19, wherein the pharmaceutical formulation results in less incidence of nausea and vomiting and better gastrointestinal (GI) tolerance compared to albiglutide when both are administered once monthly.

34. The method of claim 19, wherein the pharmaceutical formulation provides a minimal serum concentration of about 27 nanomolar throughout the treatment once a steady state is established after administration.
